(19) United States
(12) Patent Application Publication
(10) Pub. No.: US 2024/0075099 A1
(43) Pub. Date: Mar. 7, 2024

(54) COMPOSITIONS, ASSAYS, AND METHODS FOR TARGETING HDM2 AND HDMX TO REVERSE THE INHIBITION OF p53 IN PEDIATRIC CANCERS

(71) Applicant: Dana-Farber Cancer Institute, Inc., Boston, MA (US)

(72) Inventors: Loren D. Walensky, Newton, MA (US); Kimberly Stegmaier, Jamaica Plain, MA (US); Ann Morgan Cathcart, Cambridge, MA (US); Björn Stolte, München (DE)

(21) Appl. No.: 18/315,725

(22) Filed: May 11, 2023

Related U.S. Application Data
(63) Continuation of application No. 17/937,173, filed on Sep. 30, 2022, now abandoned.
Application No. 17/937,173 is a division of application No. 16/083,638, filed on Sep. 10, 2018, now abandoned.
(60) Provisional application No. 62/312,354, filed on Mar. 23, 2016.

(51) Int. Cl. A61K 38/17; A61K 31/496; A61K 45/06; A61P 35/02; C07K 4/12; C07K 14/47; G01N 33/574

(52) U.S. Cl. CPC A61K 38/1758; A61K 31/496; A61K 45/06; A61P 35/02; C07K 4/12; C07K 14/47; C07K 14/4746; G01N 33/574; G01N 33/57426; G01N 2800/52

(57) ABSTRACT

Methods for assessing the efficacy of internally cross-linked p53 transactivation domain-based inhibitor peptides in the treatment of pediatric cancer and methods of using such peptides to treat pediatric cancer are provided.

### CROSS-REFERENCE TO RELATED APPLICATIONS

This application is a continuation of U.S. application Ser. No. 17/937,173, filed Sep. 30, 2022, now abandoned, which is a divisional of U.S. application Ser. No. 16/083,638, filed Sep. 10, 2018, now abandoned, which is the National Stage of International Appl. No. PCT/US2017/023761, filed Mar. 23, 2017, which claims priority to U.S. Provisional Appl. No. 62/312,354, filed Mar. 23, 2016, the contents of each of which are incorporated by reference herein in their entirety.

### SEQUENCE LISTING

This document includes a sequence listing in electronic format submitted to the United States Patent and Trademark Office via the electronic filing system. The XML file, which is incorporated-by-reference herein, is titled “00530-0328002_SL_ST26.XML,” was created on Sep. 29, 2022, and has a size of 34,472 bytes.

### TECHNICAL FIELD

This disclosure relates to compositions, assays, methods for applying internally cross-linked (ICL) p53 transactivation domain-based inhibitor peptides (PTAIB) (targeting HDM2 and HDMX) to the treatment of pediatric cancer, and methods of predicting the efficacy of an ICL PTAIB in reversing the inhibition of p53 in pediatric cancer cells.

### BACKGROUND OF THE INVENTION

Cancer remains the second leading cause of death in children aged 5-15 years old and is the leading cause of death by a disease in children past infancy. For example, leukemia remains the leading cause of cancer-related death in children aged 1-4 years, despite significant progress in its treatment. Whereas cure rates can exceed 85% for children treated with combination chemotherapy for acute lymphoblastic leukemia (ALL) [25], there remains an urgent need to improve outcomes for children with difficult to treat or refractory forms of pediatric cancer, including acute myelogenous leukemia (AML), acute lymphoblastic leukemia (ALL), retinoblastoma, neuroblastoma, Ewing sarcoma, osteosarcoma, rhabdomyosarcoma, gliomas, and malignant rhabdoid tumor. Statistics are especially bleak for patients with relapsed disease. Thus, new therapeutic strategies are required to treat/combat refractory or relapsed pediatric cancers, including AML, for which cure rates have lagged. Surprisingly, many pediatric tumor cells (including pediatric AML, ALL, retinoblastoma, neuroblastoma, Ewing sarcoma, osteosarcoma, rhabdomyosarcoma, gliomas, and malignant rhabdoid tumor cells) retain the wild-type and/or functional form of the p53 protein, a powerful tumor suppressor, providing an opportunity to restore its anti-cancer function by targeting its negative regulators.

The p53 tumor suppressor protein plays a pivotal role in the control of a wide variety of cellular functions [1]. The prominence of p53 as “the guardian of the genome” is largely due to its ability to protect the cell from detrimental conditions such as DNA damage or starvation. Under cellular duress, p53 initiates the execution of a signaling cascade that prompts the cell to undergo arrest and allow for the repair of damaged DNA [2]. If the damage to the cell is too overwhelming, p53 promotes the transcription of genes involved in apoptosis, thus eliminating the opportunity for a compromised cell to propagate. Because p53 mediates the function of several critical control points involved in cellular homeostasis, subjugation of p53 is a common pathogenic and resistance mechanism in many cancer cells. In the context of cancer treatment, a fully operational p53 signaling system is necessary for the pro-apoptotic properties of many common chemotherapeutic agents, and a dysfunctional p53 response gives rise to chemoresistant disease [3]. Cancer cells disable wild-type and/or functional p53 by deletion [4], mutation [5], degradation [6], and/or sequestration [7]. In pediatric AML cells and other pediatric cancers, wild-type and/or functional p53 status is largely preserved, which led us to hypothesize that p53 is suppressed by other proteins in those cells. Indeed, the cellular availability of p53 is regulated by the oncoproteins HDM2 and HDMX [8]. In other words, AML and other pediatric cancer cells tolerate p53 expression because they instead overproduce HDM2 and HDMX, which effectively neutralize the anti-cancer activity of p53. These proteins latch onto a single coiled domain of p53 to either destroy or sequester it. But while the largely similar domain structures of HDM2 and HDMX allow them to bind endogenous p53 [9], their mechanisms of p53 suppression are distinct.

HDM2 targets p53 for proteasomal degradation by ubiquitylation [10], while HDMX sequesters p53 and blocks its transcriptional activity [11, 12]. See FIG. 1.

In cancers where natural or functional p53 activity has been reduced or lost, restoration of p53 activity is a strategy for cancer therapy (see, e.g., Brown et al., Nat. Rev. Cancer, 9:862-873 (2009)). The determination of the crystal structure of the p53-HDM2 binding interface contributed to the development of such strategies, e.g., by revealing that a hydrophobic cleft on the N-terminal surface of the E3 ubiquitin ligase HDM2 (Toledo and Wahl, Nat. Rev. Cancer, 6:909-923 (2006); Marine and Dyer, J. Cell. Sci., 120:371-378 (2007); Bartel et al., Int. J. Cancer, 117:469-475 (2005); Shvarts et al., Genomics, 43:34-42 (1997); Danovi et al., Mol. Cell. Biol., 24:5835-5843 (2004)) directly engages the amphipathic α-helix of the p53 transactivation domain (Kussie et al., Science, 274:948-953 (1996)). Consequently, small molecules and peptides that target the p53-binding pocket of HDM2 have been developed (see, e.g., Bernal et al., J. Am. Chem. Soc., 129:2456-2457 (2007); Grasberger et al., J. Med. Chem., 48:909-912 (2005); Koblish et al., Mol. Cancer Ther., 5:160-169 (2006); Kritzer et al., J. Am. Chem. Soc., 126:9468-9469 (2004); Shangary et al., Proc. Natl. Acad. Sci., U.S.A., 105:3933-3938 (2008); Vassilev et al., Science, 303:844-848 (2004); Yin et al., Angew. Chem. Int. Ed. Engl., 44:2704-2707 (2005)). One such agent is the small molecule HDM2 inhibitor, Nutlin-3 (see, e.g., Vassilev et al., Science, 303:844-848 (2004)). It has been shown using these agents that targeting HDM2 in certain tumors that express p53 (e.g., wild-type and/or functional p53) can lead to a therapeutic surge in p53 levels. Specifically, it has been shown that Nutlin-3 can trigger apoptosis in the absence of other therapeutics in certain tumors (see, e.g., Drakos et al., Clin. Cancer Res., 13:3380-3387 (2007); Tabe et al., Clin. Cancer Res., 15:933-942 (2009)). However, such effects do not occur in all tumors types. Specifically, certain tumors are resistant or more resistant than others to HDM2-targeting therapeutics. Co-expression of the E3 ubiquitin ligase HDMX with HDM2 can reduce the efficacy of HDM2 targeting agents (see, e.g., Hu et al., Cancer Res., J. Biol. Chem., 281:33030-33035 (2006); Patton et al., Cancer Res., 66:3169-3176 (2006); Wade et al., J. Biol. Chem., 281:33036-33044 (2006)).

The role of HDMX in regulating p53 dynamics has been described (see, e.g., Danovi et al., Mol. Cell. Biol., 24:5835-5843 (2004); Laurie et al., Nature, 444:61-66 (2006); Ramos et al., Cancer Res., 61:1839-1842 (2001); Wade et al., J. Biol. Chem., 281:33036-33044 (2006); Wang et al., Proc. Natl. Acad. Sci. U.S.A., 104:12365-12370 (2007)) and in vitro preliminary reports are available for several agents that target HDMX (see, e.g., Harker et al., Bioorg. Med. Chem., 17:2038-2046 (2009); Hayashi et al., Bioorg. Med. Chem., 17:7884-7893 (2009); Hu et al., Cancer Res., 67:8810-8817 (2007); Kallen et al., J. Biol. Chem., 284:8812-8821 (2009); Li et al., J. Am. Chem. Soc., 130:13546-13548 (2008); Michel et al., J. Am. Chem. Soc., 131:6356-6357 (2009); Pazgier et al., Proc. Natl. Acad. Sci. U.S.A., 106:4665-4670 (2009); Reed et al., J. Biol. Chem., 285:10786-10796 (2010)).

A series of hydrocarbon-stapled peptides have been invented by us (see, e.g., Bernal et al Cancer Cell 2010) and others (see, e.g., Chang et al PNAS 2013; Tan et al Sci Rep 2015) to target HDM2 and/or HDMX. Such stapled peptides with the ability to simultaneously block both HDM2 and HDMX in cancers bearing wild-type and/or functional p53 carry the promise of reactivating p53 tumor suppression in cancer.

### SUMMARY OF THE INVENTION

The present disclosure provides assays, compositions, methods of predicting the efficacy of an ICL PTAIB in reversing the inhibition of p53 in pediatric cancer cells, and methods of treatment of pediatric cancer.

More specifically, the document provides a method of treating a pediatric cancer, the method including administering one or more internally cross-linked (ICL) p53 transactivation domain-based inhibitor peptides (PTAIBs) to a subject with a pediatric cancer, the pediatric cancer having detectable wild-type or functional p53. The pediatric cancer can have detectable HDM2 and/or HDMX. All or some of the detectable HDM2 and/or HDMX can be complexed to wild-type or functional p53.

Moreover, the document additionally provides a method for predicting the efficacy of an internally cross-linked (ICL) p53 transactivation domain-based inhibitor peptide (PTAIB) in reversing the inhibition of p53 activity in a pediatric cancer, the method including:
a. testing a cell of a pediatric cancer for the presence of wild-type or functional p53, and
b. predicting that an ICL PTAIB that targets HDM2, HDMX, or HDM2 and HDMX would likely reverse inhibition of p53 activity in the cancer (and thereby treat the cancer) if the cell possesses wild-type or functional p53.

The method can include testing a cell of the pediatric cancer for the presence of HDM2 and/or HDMX, and predicting that an ICL PTAIB that targets HDM2, HDMX, or HDM2 and HDMX would likely reverse inhibition of p53 activity in the cancer if the cell possesses detectable wild-type or functional p53 and detectable HDM2 and/or HDMX. The method can include, if the cancer cell is found to express wild-type or functional p53 (and detectable HDM2 and/or HDMX), administering one or more ICL PTAIBs that target HDM2 and/or HDMX to the subject with the pediatric cancer. All or some of the detectable HDM2 and/or HDMX can be complexed to wild-type or functional p53.

Any of the above-described methods can include the administration of one or more ICL PTAIBs that target HDM2 and/or HDMX, one or more ICL PTAIBs that are stapled PTAIBs, SAH-p53-8, ALRN-7041, ALRN-6924, and/or SP315. The above-described methods can include the administration of one or more ICL PTAIBs described in U.S. Pat. No. 8,927,500 or US 2016/0101145 (see, e.g., Tables 1, 1a, 1b, 1c, or 1e of both publications. Both publications are incorporated by reference herein in their entirety).

Any of the above-described methods can further include treating the subject with one or more additional therapeutic regimens. The additional therapeutic regimens can include, e.g., surgery, chemotherapy, radiation therapy (e.g., ionizing radiation and/or ultraviolet light), hormone therapy, and/or immunotherapy (e.g., antibody therapy). For example, one or more ICL PTAIBs (e.g., one or more ICL PTAIBs that target HDM2 and/or HDMX) can be administered to the subject in conjunction with an effective amount of at least one established chemotherapeutic agent (e.g., actinomycin D, cyclophosphamide, doxorubicin, etoposide, and/or paclitaxel). In certain instances, the additional therapeutic regimen is a proteasome inhibitor. In certain instances, the additional therapeutic regimen is a Cereblon-targeting agent (e.g., lenalidomide, pomalidomide).

In any of the above-described methods, the pediatric cancer can include a pediatric leukemia. The pediatric leukemia can include, e.g., acute myeloid leukemia and/or acute lymphoblastic leukemia (e.g., a T cell lineage acute lymphoblastic leukemia or a B cell lineage acute lymphoblastic leukemia).

In any of the above-described methods, the pediatric cancer can include, e.g., Ewing sarcoma, retinoblastoma, neuroblastoma, osteosarcoma, a glioma (including, e.g., a diffuse interstitial pontine glioma), medulloblastoma, rhabdomyosarcoma (including, e.g., alveolar and/or embryonal rhabdomyosarcoma), Wilm's tumor, and/or a malignant rhabdoid tumor.

In any of the above-described methods, the pediatric cancer can include a relapsed cancer.

In any of the above-described methods, the pediatric cancer can be (known, predicted, and/or determined to be) refractory to one or more previous treatments (e.g., surgery, chemotherapy, radiation therapy, hormone therapy, and/or immunotherapy).

As used herein, a “wild-type gene” refers to a germ-line gene having a nucleic acid sequence that occurs in non-cancerous, somatic cells. See, e.g.,

<http://p53.iarc.fr/p53Sequences.aspx> and <http://p53.iarc.fr/p53Sequence.aspx> for exemplary human p53 wild-type gene sequences. As used herein, a “wild-type protein” refers to a protein encoded by a wild-type gene, or by a gene with one or more silent mutations or polymorphisms. Wild-type human p53 has the amino acid sequence of SEQ ID NO: 1.

As used herein, a “functional gene” is a wild-type gene or a gene having one or more mutations, as compared to the corresponding wild-type gene, that do not result in complete loss of any essential function in the protein encoded by the functional gene, as compared to the protein encoded by the corresponding wild-type gene. As used herein, a “functional protein” is a wild-type protein or a protein having one or more amino acid changes, as compared to the corresponding wild-type protein, that do not result in complete loss of any essential function in the functional protein, as compared to the corresponding wild-type protein.

As used herein, a “fully functional gene” is a wild-type gene or a gene having one or more mutations, as compared to the corresponding wild-type gene, that result in no loss of any function in the protein encoded by the fully functional gene, as compared to the protein encoded by the corresponding wild-type gene. As used herein, a “fully functional protein” is a wild-type protein or a protein having one or more amino acid changes, as compared to the corresponding wild-type protein, that result in no loss of any function in the fully functional protein, as compared to the corresponding wild-type protein.

As used herein, a cell containing “functional p53” (gene and/or protein) is a cell in which one allele or both alleles encode(s) wild-type and/or functional p53. Thus, the term includes a cell containing, e.g., p53 encoded by alleles (both or one) containing silent mutations or mutations that do not result in complete loss of all p53 function (e.g., the capacity of p53 to induce cell cycle arrest or cell death by any of its mechanisms).

As used herein, the term “gene” can be replaced with “protein-encoding nucleic acid”.

As used herein, the terms “about” and “approximately” are defined as being within plus or minus 10% of a given value or state, preferably within plus or minus 5% of said value or state.

The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment of infection).

As used herein, a “pediatric cancer” is any cancer that occurs in a pediatric subject (e.g., a “pediatric patient”) and occurs at the same frequency, or at a greater frequency, in pediatric subjects as in adult subjects. Also, as used herein, a human “pediatric subject” (e.g., a pediatric patient) is a human subject that is from newborn to 21 years of age and a human “adult subject” (e.g., an “adult patient”) is a human subject that is older than 21 years of age.

As used herein, a “p53 transactivation domain-based inhibitor peptide” (“PTAIB”) is a peptide that includes all or part of transactivation domain sequences corresponding to amino acids 14-29 of human p53 (e.g., and at least the essential interacting amino acids F19, W23, and L26) and completely or partially inhibits the binding of p53 to HDMX, HDM2, or HDMX and HDM2, as measured in an in vitro binding assay. The term “PTAIB” includes PTAIB having a wild-type and/or fully functional amino acid sequence or a wild-type and/or fully functional amino acid sequence but with one or more of the amino acids being modified as described in the section below entitled “Amino acid modifications in ICL PTAIBs”. For example, any or all amino acids except for the essential interacting amino acids (see above) can be substituted, and/or one or more of the essential interacting amino acids (see above) can be substituted with one or more conservative substitutions (as defined herein). See, e.g., Coffill et al Genes Dev 2016 30: 281-292 and Baek at el JACS 2012 13: 103-6. The human wild-type amino acid sequence of the p53 transactivation domain that engages HDM2 and HDMX includes:

(SEQ ID NO: 2)
LSQETFSDLWKLLPEN

which corresponds to amino acids 14-29 in this example.

As used herein, an internally cross-linked (ICL) PTAIB (e.g., a stapled PTAIB) has the same properties as the parent PTAIB from which it is produced but will have at least 40% (e.g., at least: 50%; 60%; 70%; 75%; 80%; 85%; 90%; 95%; 98%; 100%; or more) of the ability of the parent PTAIB to inhibit the binding of p53 to HDMX, HDM2, or HDMX and HDM2, as measured in an in vitro binding assay.

As used herein, a control level of expression of a protein (e.g., HDMX or HDM2) is the level of expression of that protein detected in a cell (referred to as a control cell) of the same tissue type as the pediatric cancer cell but from non-cancerous tissue of the same subject from which the pediatric cancer cell was obtained. Alternatively, the control cell can be of the same tissue type as the pediatric cancer cell but be from non-cancerous tissue of a subject other than that from which the pediatric cancer cell was obtained. Moreover, the control level of expression can be an average level of expression obtained by testing a plurality of cells, each cell being of the same tissue type as the pediatric cancer cell but from non-cancerous tissue of a different subject, each subject being a subject other than that from which but the pediatric cancer cell was obtained. Other methods for determining control levels of expression are well known to those in the art.

As used herein, “levels of expression” in test cells or control cells can be in terms of mRNA or protein expression. mRNA expression can be measured in a variety of ways including, e.g., reverse transcription-polymerase chain reaction (RT-PCR) assays, Northern blots, or in situ hybridization assays. Protein expression can measured by, e.g., Western blots, far Western blots, immunoprecipitation or co-immunoprecipitation assays, pull-down assays, enzyme-linked immunosorbent assays (ELISAs), metabolic labeling assays, immunocytochemical assays, or immunofluorescence assays. The data from assays and tests for level of expression can be quantitative (i.e., numerical, e.g., 2.5 micrograms, 0.05-0.2 optical density units), semi-quantitative (e.g., “+++”, “++”, “+”; “black fill”, “dark grey fill”, “light grey fill”, “white fill”), or qualitative (e.g., “+” or “−”; “present” or “absent”; “black” or “white”).

ICL PTAIBs, and PTAIBs from which ICL PTAIBs can be made employing methods known to those in the art, useful for the methods of the present document are disclosed in, e.g., U.S. Pat. Nos.6,153,391; 7,083,983; 8,609,809; 8,637,859; 8,637,686; 8,859,723; 8,927,500; 8,897,414; 9,023,988; and 9,206,223: U.S. Patent Application Publication Nos: US2001/0018511; US2005/0137137; US2013/0274205; US2014/00183002; and US2015/0246946: and the scientific articles Brown et al. (2013) ACS Chem. Biol. 8(3): 506-512; Yurlova et al. (2014) J. Biomol. Screen 19(4): 516-525; Khoo et al. (2014) Nat. Rev. Drug Discov. 13(3): 217-236; Sim et al. (2014) J. Chem. Theory Comput. 1753-1761; Lau et al. (2014) Org. Biomol. Chem. 12(24): 4074-4077; Chee et al. (2014) PloS One 9(8): e104914; Tan et al. (2015) Sci. Rep. 5:12116; and ElSawy et al. (2016) J. Phys. Chem. B 120(2): 320-328, the disclosures of which are incorporated herein by reference in their entirety.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIG. 1 is a depiction of HDM2 and/or HDMX-mediated suppression of the p53 tumor suppressor pathway.

FIG. 2A and FIG. 2B are graphs depicting the expression of HDM2 (FIG. 2A) and HDMX (FIG. 2B) in the cancer cell line encyclopedia. Highlighted by arrows are AML cell lines. Expression levels: low ≤5, medium 5-8, high >8. From bottom to top, the Y-axis labels for FIG. 2A are: 5, 6, 7, 8, 9, 10, 11. The entire Y-axis for FIG. 2A is labeled log2 RNA expression level. From left to right, the X-axis labels for FIG. 2A are: B-cell ALL (15); lymphoma Burkitt (11); meningioma (9); kidney (34); lymphoma other (29); lymphoma Hodgkin (12); prostate (7); melanoma (61); lymphoma DLBCL (18); multiple myeloma (80); other (20); medulloblastoma (4); lung small cell (53); neuroblastoma (17); T-cell ALL (15); breast (58); urinary tract (27); osteosarcoma (10); Ewing's sarcoma (10); soft tissue (21); endometrium (27); mesothelioma (11); stomach (88); AML (84); colorectal (61); lung NSC (130); CML (14); esophagus (25); ovary (51); pancreas (44); bile duct (8); liver (28); thyroid (12); upper aerodigestive (32); glioma (62); chondrosarcoma (4). From bottom to top, the Y-axis labels for FIG. 2B are: 6, 7, 8, 9. The entire Y-axis for FIG. 2B is labeled log2 RNA expression level. From left to right, the X-axis labels for FIG. 2B are: B-cell ALL (15); lymphoma Burkitt (11); T-cell ALL (15); CML (14); AML (34); neuroblastoma (17); lymphoma DLBCL (16); lymphoma other (29); medulloblastoma (4); multiple myeloma (30); Ewing's sarcoma (10); lung small cell (68); breast (58); prostate (7); endometrium (27); lymphoma Hodgkin (12); thyroid (12); colorectal (61); bile duct (6); stomach (36); urinary tract (27); pancreas (44); soft tissue (21); kidney (34); other (20); liver (28); osteosarcoma (10); ovary (51); lung NSC (130); melanoma (61); esophagus (25); upper aerodigestive (32); meningioma (8); glioma (62); mesothelioma (11); chondrosarcoma (4). FIG. 2C and FIG. 2D are Z-score depletion graphs of 11,194 dependencies in the MOLM-13 AML cell line (FIG. 2C) and the MV4; 11 AML cell line (FIG. 2D). HDMX ranks as #1 of 11,194 dependencies in the MOLM-13 AML cell line and #3 of 11,194 in the MV4; 11 AML cell line. In contrast, HDM2 ranks as #6002 and #1324 of 11,194 dependencies in the MOLM-13 and MV4; 11 cell lines, respectively. HDMX and HDM2 rankings are indicated with respect to all shRNA rankings for each cell line.

FIG. 3A-FIG. 3E is a series of depictions of the pharmacologic blueprint for reactivating the p53 pathway based on the cancer cell's p53-HDM2-HDMX axis and interaction dynamics.

FIG. 4A-FIG. 4B are structural models of HDM2 (FIG. 4A) and HDMX (FIG. 4B) complexes with hydrocarbon-stapled p53 peptides. PDB ID: 3V3B (4A), 4N5T (FIG. 4B).

FIG. 5A-FIG. 5C is a series of line graphs depicting the potent and sequence-dependent binding activity of FITC-SAH-p53-8 for HDMX (FIG. 5A), and showing that whereas Nutlin-3 is only capable of dissociating the FITC-SAH-p53-8/HDM2 complex (FIG. 5B), SAH-p53-8 effectively disrupts the association of FITC-SAH-p53-8 with both HDM2 and HDMX (FIG. 5B, FIG. 5C). The dark dots at the bottom of the graph in FIG. 5A represents FITC-WT p5314-29; an arrow points to FITC-SAH-p53-8; another arrow points to FITC-SAH-p53-8_{F19A}. From left to right, the X-axis for FIG. 5A is labeled 10⁻¹⁰, 10⁻⁹, 10⁻⁸, 10⁻⁷. The entire Y-axis for FIG. 5A is labeled HDMX Direct Binding (mP). The entire X-axis for FIG. 5A is labeled [HDMX] M. An arrow in FIG. 5B represents SAH-p53-8 (IC₅₀=218±48 nM); another arrow points represents Nutlin-3 (IC₅₀=2.11±0.62 μM). From left to right, the X-axis for FIG. 5B is labeled 10⁻⁸, 10⁻⁷, 10⁻⁶, 10⁻⁵. The entire Y-axis for FIG. 5B is labeled HDM2 Composition (mP). The entire X-axis for FIG. 5B is labeled [Compound] M. An arrow in FIG. 5C represents SAH-p53-8 (IC₅₀=229±57 nM); another arrow represents Nutlin-3 (IC₅₀>10 μM). From left to right, the X-axis for FIG. 5C is labeled 10⁻⁹, 10⁻⁸, 10⁻⁷, 10⁻⁶, 10⁻⁵. The entire Y-axis for 5A is labeled HDMX Composition (mP). The entire X-axis for FIG. 5A is labeled [HDMX] M.

FIG. 6A-FIG. 6C is a series of graphs showing that the dual HDM2/HDMX inhibitor, ALRN-7041, dose-responsively impairs the viability of a pediatric AML cell line, which is otherwise resistant to the selective HDM2 inhibitor, Nutlin-3a (FIG. 6A). Importantly, single point mutagenesis at the interacting surface of ALRN-7041 abrogates the cytotoxic effect, highlighting the specificity of action. ALRN-7041 and its mutant control, ALRN-7342, are both readily taken up by cells in the presence of full serum (FIG. 6B) and without membrane disruption (FIG. 6C), as measured by fluorescence scan of lysates from treated cells (4 h) and LDH release (30 min). These data underscore the selectivity of ALRN-7041 cytotoxicity in MV4; 11 cells (FIG. 6A).

FIG. 7 is a depiction of various exemplary stapled p53 peptide compositions. Modifications to the wild type sequence are shown in lighter shade. Z, cyclobutylalanine (Cba); *, stapling amino acid positions. SEQ ID NOs: 8, 3, 9, 5, 11-13 from top to bottom, respectively.

FIG. 8A, FIG. 6B are Western blot images depicting the dissociation of the anti-apoptotic p53/HDMX complex by a stapled p53 peptide. FIG. 8A shows that a stapled p53 peptide (SAH-p53-8), but not Nutlin-3, dissociates the p53/HDMX complex in cancer cells, as measured by co-immunoprecipitation. FIG. 8B shows the dose-responsive dissociation of p53/HDMX by SAH-p53-8.

FIG. 9 is a dot plot depicting MDM2 dependency in Ewing sarcoma in a CRISPR screen. Data is plotted as z-score (x-axis) versus scaled rank (y-axis). Dark dots show Ewing sarcoma cell lines. TP53 wild type Ewing sarcoma cell lines TC32 and CADOES1 are denoted with arrows.

FIG. 10 is a dot plot depicting MDM4 dependency in Ewing sarcoma in a CRISPR screen. Data is plotted as z-score (x-axis) versus scaled rank (y-axis). Dark dots show Ewing sarcoma cell lines. p53 wild type cell lines TC32 and CADOES1 are denoted with arrows.

FIG. 11 is a series of graphs showing that selective susceptibility of pediatric leukemia cell lines to ALRN-7041 is based on wild-type p53 expression.

FIG. 12 depicts the results of flow cytometry studies showing that ALRN-7041 dose-responsively upregulates p53 protein level in RS4; 11 cells.

FIG. 13 provides graphs illustrating the susceptibility of pediatric diffuse interstitial pontine glioma (DIPG) neurospheres to ALRN-7041.

FIG. 14 depicts graphs showing that Ewing sarcoma cell lines bearing wild-type p53 are selectively susceptible to ALRN-7041 treatment.

FIG. 15 provides the results of Western blot analyses of electrophoresed lysates from p53 wild-type TC32 and TC138 Ewing sarcoma cells treated with ALRN-7041 at the indicated doses and time points and probed with anti-MDM2, p53, and p21 antibodies.

FIG. 16 compares the effect of ALRN-7041 relative to DMSO on apoptosis in a Ewing sarcoma cell line bearing wild-type p53.

FIG. 17 shows the results of a western blot analysis of ALRN-7041 treatment of mice bearing a TC32 Ewing Sarcoma xenograft on MDM2, p53, and p21 protein levels in tumor tissue.

FIG. 18 are bar graphs depicting the effect of ALRN-7041 treatment of mice bearing TC32 Ewing Sarcoma xenografts on MDM2 and p21 mRNA levels in tumor tissue.

FIG. 19 is a graphical depiction of the effect of treatment of mice bearing TC32 Ewing Sarcoma xenografts with 30 mg/kg ALRN-7041 IV q.o.d. (grey) or vehicle (black) on tumor growth.

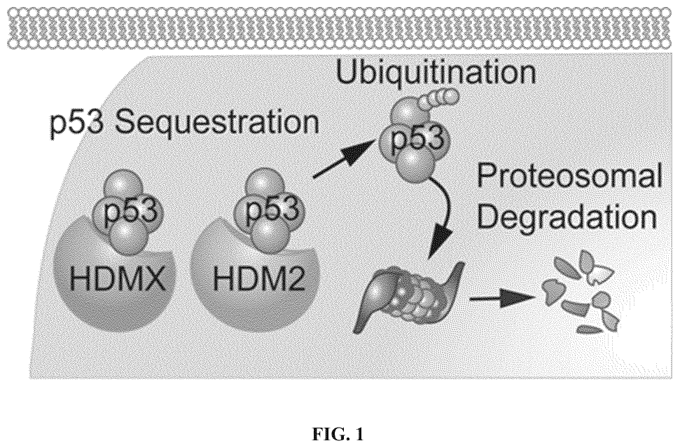

### DETAILED DESCRIPTION

This disclosure is based on the finding that internally cross-linked p53 transactivation domain-based inhibitor peptides show cytotoxicity across a spectrum of pediatric cancer types.

This document provides methods of treating a pediatric cancer in a human subject in need thereof by administering to the human subject a therapeutically effective amount of an internally cross-linked (e.g., stapled or stitched) p53 transactivation domain-based inhibitor peptide. The internally cross-linked p53 transactivation domain-based inhibitor peptide can comprise a “cap” at the N-terminal and/or C-terminus. In some cases, the internally cross-linked p53 transactivation domain-based inhibitor peptide further comprises an acetyl group at the N-terminus of the peptide. In some cases, the internally cross-linked p53 transactivation domain-based inhibitor peptide further comprises a CONH2 (amide) group at the C-terminus of the peptide. In certain cases, the internally cross-linked p53 transactivation domain-based inhibitor peptide further comprises an acetyl group at the N-terminus of the peptide and CONH2 (amide) group at the C-terminus of the peptide. In certain cases, the internally cross-linked p53 transactivation domain-based inhibitor peptide is SAH-p53-8. In other cases, the internally cross-linked p53 transactivation domain-based inhibitor peptide is ALRN-6924. In yet other cases, the internally cross-linked p53 transactivation domain-based inhibitor peptide is SP315. In certain cases, the internally cross-linked p53 transactivation domain-based inhibitor peptide is not SAH-p53-8 or SP315. In certain cases, the internally cross-linked p53 transactivation domain-based inhibitor peptide is a cross-linked peptide described in U.S. Pat. No. 8,927,500 and US 2016/0101145 (e.g., a peptide listed in Table 1, Table 1a, Table 1b, Table 1c, or Table le of both publications) (this US patent and US patent publication are incorporated by reference herein in their entireties). In one case, the internally cross-linked p53 transactivation domain-based inhibitor peptide comprises the amino acid sequence: LTFX₁EYWAQZX₂SAA, wherein X₁ and X₂ are non-natural amino acids (e.g., R-octenyl alanine, S-pentenyl alanine) that can be cross-linked to form a hydrocarbon staple, and Z is a leucine mimetic (e.g., cyclobutylalanine (Cba)) (SEQ ID NO:6). In some cases, this internally cross-linked p53 transactivation domain-based inhibitor peptide further comprises an acetyl group at the N-terminus of the peptide. In some cases, this internally cross-linked p53 transactivation domain-based inhibitor peptide further comprises a CONH2 (amide) group at the C-terminus of the peptide. In certain cases, this internally cross-linked p53 transactivation domain-based inhibitor peptide further comprises an acetyl group at the N-terminus of the peptide and CONH2 (amide) group at the C-terminus of the peptide. In certain instances, X₁ and X₂ are the same non-natural amino acids; in other cases, X₁ and X₂ are different non-natural amino acids. In some cases, X₁ and X₂ are independently R8 (R-octenyl alanine) or S5 (S-pentenyl alanine). In some cases, X₁ is R8 and X₂ is S5. In other cases, X₁ is S5 and X₂ is R8. In another case, the internally cross-linked p53 transactivation domain-based inhibitor peptide comprises the amino acid sequence that is identical to LTFX₁EYWAQZX₂SAA (SEQ ID NO:6), except having 1-9 amino acid substitutions (e.g., 1, 2, 3, 4, 5, 6, 7, 8, or 9). These substitutions may be conservative or non-conservative. In certain embodiments, F, W, and Z in this sequence are not substituted. In certain embodiments, Z in this sequence is substituted with leucine. In certain instances, the internally cross-linked p53 transactivation domain-based inhibitor peptide is 14 to 100 amino acids (counting both natural and non-natural amino acids) in length. In other instances, the internally cross-linked p53 transactivation domain-based inhibitor peptide is 14 to 50 amino acids (counting both natural and non-natural amino acids) in length. In yet other instances, the internally cross-linked p53 transactivation domain-based inhibitor peptide is 14 to 25 amino acids (counting both natural and non-natural amino acids) in length. In some other instances, the internally cross-linked p53 transactivation domain-based inhibitor peptide is 14 to 20 amino acids (counting both natural and non-natural amino acids) in length. The human subject may be an infant (new born to 1-year old), or a child of 1 to 18 years of age. In certain instances, the child is between 5 and 15 years of age. The cancer cells of the human subject to be treated comprise wild type p53 protein or functional p53 protein. The cancer cells of the human subject to be treated also comprise HDM2 and/or HDMX. In the cancer cells of the human subject to be treated, at least some (e.g., 5%, 10%, 20%, 25%, 30%, 40%, 50%) of the HDM2 and/or HDMX are complexed with p53 protein. In some cases, the pediatric cancer is a refractory form of pediatric cancer. In certain instances, the pediatric cancer is pediatric acute myelogenous leukemia (AML), acute lymphoblastic leukemia (ALL), retinoblastoma, neuroblastoma, Ewing sarcoma, osteosarcoma, rhabdomyosarcoma, glioma (e.g., interstitial pontine glioma), or malignant rhabdoid tumor. In certain embodiments, a therapeutically effective amount of an internally cross-linked p53 transactivation domain-based inhibitor peptide is 0.1 mg/kg to 200 mg/kg of the cross-linked peptide. In other embodiments, a therapeutically effective amount of an internally cross-linked p53 transactivation domain-based inhibitor peptide is 1 mg/kg to 150 mg/kg of the cross-linked peptide. In yet other embodiments, a therapeutically effective amount of an internally cross-linked p53 transactivation domain-based inhibitor peptide is 5 mg/kg to 100 mg/kg of the cross-linked peptide. In yet other embodiments, a therapeutically effective amount of an internally cross-linked p53 transactivation domain-based inhibitor peptide is 10 mg/kg to 50 mg/kg of the cross-linked peptide. In certain instances, the treatment involves administering the internally cross-linked p53 transactivation domain-based inhibitor peptide in combination with another agent(s) that are useful in treating the pediatric cancer. In certain cases, the agent is a proteasomal inhibitor. In certain cases, the agent is a Cereblon-targeting agent. In certain cases, the agent is lenalidomide and/or pomalidomide. In some cases, the treatment involves administering the internally cross-linked p53 transactivation domain-based inhibitor peptide in combination with chemotherapy or radiotherapy.

### Definitions

A “non-essential” amino acid residue is a residue that can be altered from the wild-type and/or fully functional sequence of a polypeptide (without abolishing or substantially altering its activity). An “essential” amino acid residue is a residue that, when altered from the wild-type and/or fully functional sequence of the polypeptide, results in abolishing or substantially abolishing the polypeptide activity.

In some embodiments, the term “essential” amino acid residue as used herein, includes conservative substitutions of the essential amino acid. Generally, the “essential” amino acid residues are found at the interacting face of the alpha helix.

The term “amino acid side chain” refers to a moiety attached to the α-carbon in an amino acids. For example, the amino acid side chain for alanine is methyl, the amino acid side chain for phenylalanine is phenylmethyl, the amino acid side chain for cysteine is methylthiol, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an alpha di-substituted amino acid).

The term “polypeptide” encompasses two or more naturally occurring or synthetic amino acids linked by a covalent bond (e.g., an amide bond). Polypeptides as described herein include full length proteins (e.g., fully processed proteins) as well as shorter amino acids sequences (e.g., fragments of naturally occurring proteins or synthetic polypeptide fragments).

The term “halo” refers to any radical of fluorine, chlorine, bromine or iodine. The term “alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. In the absence of any numerical designation, “alkyl” is a chain (straight or branched) having 1 to 20 (inclusive) carbon atoms in it. The term “alkylene” refers to a divalent alkyl (i.e., —R—).

The term “alkenyl” refers to a hydrocarbon chain that may be a straight chain or branched chain having one or more carbon-carbon double bonds in either Z or E geometric configurations. The alkenyl moiety contains the indicated number of carbon atoms. For example, C₂-C₁₀ indicates that the group may have from 2 to 10 (inclusive) carbon atoms in it. The term “lower alkenyl” refers to a C₂-C₈ alkenyl chain. In the absence of any numerical designation, “alkenyl” is a chain (straight or branched) having 2 to 20 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂-C₁₀ indicates that the group may have from 2 to 10 (inclusive) carbon atoms in it. The term “lower alkynyl” refers to a C₂-C₈ alkynyl chain. In the absence of any numerical designation, “alkynyl” is a chain (straight or branched) having 2 to 20 (inclusive) carbon atoms in it.

The term “aryl” refers to a 6-carbon monocyclic or 10-carbon bicyclic aromatic ring system wherein 0, 1, 2, 3, 4, or 5 atoms of each ring may be substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally may be optionally substituted. Preferred cycloalkyl groups include, without limitation, cyclopropyl, cyclobutyl, cyclopentyl, cyclopentenyl, cyclohexyl, cyclohexenyl, cyclohexadienyl, cycloheptyl, cycloheptadienyl, cycloheptatrienyl, cyclooctyl, cyclooctenyl, cyclooctadienyl, cyclooctatrienyl, and cyclooctynyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyrrolyl, pyridyl, furyl or furanyl, imidazolyl, 1,2,3-triazolyl, 1,2,4-triazolyl, benzimidazolyl, pyridazyl, pyrimidyl, thiophenyl, quinolinyl, indolyl, thiazolyl, oxazolyl, isoxazolyl and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, aziridinyl, oxiryl, thiiryl, morpholinyl, tetrahydrofuranyl, and the like.

The term “substituents” refers to a group “substituted” on an alkyl, cycloalkyl, aryl, heterocyclyl, or heteroaryl group at any atom of that group. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, azido, and cyano groups.

### Stapled Peptides

In the peptide sequences disclosed herein, the symbol “a” represents D-alanine, an “*” denotes the location of an all-hydrocarbon staple, an “—NH₂” at the C-terminus of a sequence indicates that the C-terminal amino acid is amidated, a “$” or “$r8” indicates that the residue can be substituted with a residue capable of forming a crosslinker with a second residue in the same molecule or a precursor of such a residue, and an “Ac” represents an acetyl group.

SAH-p53-8 comprises the following sequence:

| (SEQ ID NO: 3) | |
|---|---|
| | Ac—QSQQTF*NLWRLL*QN—NH₂ |

In comparison, wild-type p53 comprises the following sequence between amino acids 14-29:

(SEQ ID NO: 2)
LSQETFSDLWKLLPEN

As another example, another ICL PTAIB, ALRN-6924 (<ClinicalTrials.gov>identifier: NCT02264613 and NCT02909972) is currently undergoing clinical trials.

SP315 (see, e.g., U.S. Pat. No. 8,927,500) is another example of an ICL PTAIB.

SP315 comprises the following sequence:

(SEQ ID NO: 4)
Ac-LTF$AYWAQL$AAAAAa-NH₂

### ALRN-7041

An optimized ICL PTAIB, ALRN-7041, having improved drug-like properties for engagement of HDM2 and HDMX in cells and in vivo has been developed. ALRN-7041 thus has the potential to restore p53-mediated apoptosis in pediatric cancers that retain functional p53 coincident with expression of HDM2 and/or HDMX (including, e.g., AML), positioning it or its next-generation analogs, such as SP315, to become the very first stapled peptide therapeutics for treating these cancers. Such ICL PTAIBs represent a new chemical modality for specifically targeting pathologic protein interactions in human cancers, including pediatric cancers that retain functional p53 coincident with expression of HDM2 and/or HDMX.

ALRN-7041 comprises the following sequence:

(SEQ ID NO: 5)
Ac-LTF*EYWAQZ*SAA-NH₂

In particular, ALRN-7041 was generated by installing ani, i+7 all-hydrocarbon staple at positions S20 and P27 of the p53 transactivation domain-based inhibitor peptide helix, the same staple location determined originally by the inventors of SAH-p53-8 (see, e.g., Bernal et al. (2007) JACS, Bernal et al. (2010) Cancer Cell). In accordance with our specifications in “Amino acid modifications in ICL PTAIBs”, amino acid substitutions within the p53 transactivation domain sequences of ALRN-7041 were made in conserved and non-conserved areas based on phage-display sequence optimization against the targets (see, e.g., Pazgier et al. (2009) PNAS 106:4665-4670). Additional residues on the non-interacting face of the helix were also modified to improve peptide solubility and cellular uptake. (ICL PTAIBs are synthesized by replacing two naturally occurring amino acids with the non-natural S-octenyl and R-pentenyl alanines at discrete locations flanking, e.g., 6 amino acids (e.g., in this case, the i, i+7 positions). To synthesize PTAIBs, we used solid phase Fmoc chemistry and ruthenium-catalyzed olefin metathesis, followed by peptide deprotection and cleavage, purification by reverse phase high performance liquid chromatography/mass spectrometry (LC/MS), and quantification by amino acid analysis. N-termini were capped with acetyl, FITC, or biotin.

The invention features a modified polypeptide (i.e., an ICL PTAIB) of Formula (I),

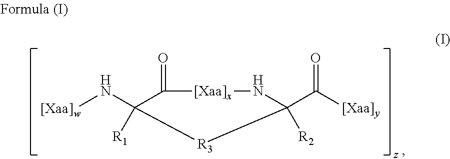

or a pharmaceutically acceptable salt thereof,

wherein;

each R₁ and R₂ are independently H or a C₁ to C₁₀ alkyl, alkenyl, alkynyl, arylalkyl, cycloalkylalkyl, heteroarylalkyl, or heterocyclylalkyl;

each R₃ is alkylene, alkenylene or alkynylene (e.g., a C₆, C₇, or C₁₁ alkenylene) substituted with 1-6 R₄;

each R₄ is, independently —NH₃ or —OH, wherein each —NH₃ is optionally substituted;

wherein each R₃ replaces, relative to the corresponding parent (i.e., unmodified) non-internally cross-linked PTAIB, the side chains of at least one pair (e.g., one or two pairs) of amino acids separated by 2, 3, or 6 amino acids (i.e., x=2, 3, or 6).

As used above, and elsewhere in the present document, a “corresponding parent p53 transactivation domain-based inhibitor peptide (PTAIB)” can be a wild-type and/or fully functional PTAIB, or any of the variants of a wild-type and/or fully functional PTAIB disclosed in the present document, except that such a variant would not include an internal cross-link as described herein.

In the case of Formula I, the following embodiments are among those disclosed.

In cases where x=2 (i.e., i+3 linkage), R₃ can be a C₇ alkylene or alkenylene. Where it is an alkenylene, there can one or more double bonds. In cases where x=6 (i.e., i+4 linkage), R₃ can be a C₁₁, C₁₂, or C₁₃ alkylene or alkenylene. Where it is an alkenylene, there can one or more double bonds. In cases where x=3 (i.e., i+4 linkage), R₃ can be a C₈ alkylene or alkenylene. Where it is an alkenylene, there can one or more double bonds.

In certain instances, the two alpha, alpha disubstituted stereocenters (alpha carbons) are both in the R configuration or S configuration (e.g., i, i+4 cross-link), or one stereocenter is R and the other is S (e.g., i, i+7 cross-link). Thus, where Formula I is depicted as

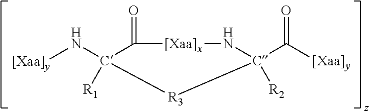

the C′ and C″ disubstituted stereocenters can both be in the R configuration or they can both be in the S configuration, e.g., when x is 3. When x is 6, the C′ disubstituted stereocenter is in the R configuration and the C″ disubstituted stereocenter is in the S configuration or the C′ disubstituted stereocenter is in the S configuration and the C″ disubstituted stereocenter is in the R configuration. The R₃ double bond (based on the definition above, R₃ contains an alkane, alkene, or alkyne moiety; in general, it is an alkene) may be in the E or Z stereochemical configuration. Similar configurations are possible for the carbons in Formula II corresponding to C′ and C″ in the formula depicted immediately above.

In some embodiments, ICL PTAIBs can include (e.g., comprise, consist, or consist essentially of) amino acid sequences related or with identity to a portion or portions of the wild type and/or fully functional human p53 protein or amino acid sequence (e.g., SEQ ID NO: 1). Alternately or in addition, ICL PTAIBs can include amino acid sequences related or with identity to a portion or portions of the wild-type and/or fully functional protein or amino acid sequence of p53 in one or more non-human animals, including, e.g., jawed vertebrates (gnathostomes) (including, e.g., cartilaginous fish, ray-finned fish, lobe-finned fish, amphibians, reptiles, birds, and mammals) and jawless vertebrates (cyclostomes) (including, e.g., lampreys and hagfish). For example, peptides can include one or more domains of wild-type and/or fully functional p53, e.g., the p53 transactivation domain. Such domains can be naturally contiguous. Alternatively, non-naturally contiguous p53 domains can be combined. In some instances, peptides can include at least six (e.g., 7, 8, 9, 10, 11, 12, 13, 14, 15, 16, 17, 18, 19, 20, 25, 30, 35, 40, 45, 50 amino acids, or any number between 20-50 amino acids, or any range between any two of the recited number of amino acids) amino acids of SEQ ID NO: 1. The amino acids are contiguous except that one or more pairs of amino acids separated by 2, 3, or 6 amino acids are replaced by amino acid substitutes that form a cross-link, e.g., via R₃. Thus, at least two amino acids can be replaced by tethered amino acids or tethered amino acid substitutes.

The peptides can include 8 (9, 10, 11, 12, 13, 14, 15, 16, 17, 18, 19, 20, 21, 22, 23, 24, 25, 30, 35, 40, 45, 50 or more) contiguous amino acids of a p53 polypeptide (e.g., SEQ ID NOs: 1 or 2) wherein the alpha carbons of two amino acids that are separated by three amino acids (or six amino acids) are linked via R₃, one of the two alpha carbons is substituted by R₁ and the other is substituted by R₂ and each is linked via peptide bonds to additional amino acids.

### Amino Acid Modifications in ICL PTAIBs

In some instances, ICL PTAIBs with identity to a portion or portions of SEQ ID NO: 1 can have a first level of identity for amino acids corresponding to amino acids in the interacting face of p53 (e.g., the interacting face of the transactivation domain of p53) and a second level of identity for amino acids not corresponding to the interacting face. For example, amino acids corresponding to amino acids in the interacting face of p53 (e.g., the interacting face of the transactivation domain of p53) can be conserved or can be conservative substitutions of the amino acids present in the interacting face of p53 (e.g., the interacting face of the transactivation domain of p53). In contrast, amino acids outside the interacting face can have at least or about 30%, at least or about 40%, at least or about 50%, at least or about 60%, at least or about 70%, at least or about 80%, at least or about 90%, at least or about 95%, at least or about 98%, at least or about 99%, or 100% identity to those amino acids outside the interacting face of the peptide). Alternatively or in addition, amino acids outside those in the interacting face can include amino acid substitutions and/or deletions, whether conservative or not. For example, amino acids outside those in the interacting face can include 1, 2, 3, 4, 5, 6, 7, 8, less than 10, less than 5, less than 4, less than 3, or less than 2 amino acid substitutions, deletions, and/or additions, whether conservative or not.

The “interacting face” of the ICL PTAIBs includes those amino acid residues of the p53 alpha helix that interact (e.g., interact specifically or bind specifically) with HDM2 and/or HDMX. Amino acid residues contained within the interacting face of p53, including amino acid residues contained within the interacting face of the p53 transactivation domain, are known in the art (see, e.g., Kussie et al., Science, 274(5289):948-953 (1996), and Joseph et al., Cell Cycle, 9(22):4560-4568 (2010)). In some instances, amino acids of peptides disclosed herein that correspond to amino acids within the interacting face of p53 as disclosed by, e.g., Kussie et al., Science, 274(5289):948-953 (1996) or Joseph et al., Cell Cycle, 9(22):4560-4568 (2010) can be the same or conservative substitutions of the amino acids disclosed by, e.g., Kussie et al., Science, 274(5289):948-953 (1996) and Joseph et al., Cell Cycle, 9(22):4560-4568 (2010). For example, in some instances, amino acids in the interacting face of the peptides disclosed herein correspond to Phe19, Trp23, and Leu26 of wild type p53 (SEQ ID NO: 1). Conservative substitutions suitable for inclusion in the peptides disclosed herein are discussed below. For example, in some instances, a “conservative amino acid substitution” can include substitutions in which one amino acid residue is replaced with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In some instances, in the context of amino acids in the interacting face of the peptides disclosed herein, a conservative amino acid substitution is an amino acid substitution that does not change the structure of the hydrophobic interacting face of the peptide. For example, a conservative amino acid substitution is an amino acid substitution that does not reduce (e.g., substantially reduce) binding of the peptide to HDM2 and/or HDMX. Methods for detecting any reduction in binding can include comparing binding affinity following conservative amino acid substitution, wherein any amino acid substitution that reduces (e.g., substantially reduces) binding are not conservative amino acid substitutions. In some embodiments, substantially reduced binding can include binding that is 10%, 20%, 30%, 40%, 50%, 60%, 70%, 80%, 90%, 95%, 98%, 99%, or 100% less than binding of the unmodified peptide to HDM2 and/or HDMX. Methods for assessing interaction between a peptide and HDM2 and/or HDMX are disclosed herein. Methods for identifying the interactive face of a peptide are known in the art (see, e.g., Broglia et al., Protein sci., 14(10):2668-81, 2005; Hammond et al., J. Pharm. Sci., 98(1):4589-603, 2009; Ng and Yang, J. Phys. Chem. B., 111(50):13886-93, 2007; and Bird et al., PNAS USA, 197:14093, 2010).

In some embodiments, as indicated above, amino acid sequences of the ICL PTAIBs herein can vary outside of those amino acids corresponding to the interacting face (e.g., Phe6, Trpio, and/or Leui3) almost without limitation. For example, amino acids outside those in the interacting face can have at least or about 30%, at least or about 40%, at least or about 50%, at least or about 60%, at least or about 70%, at least or about 80%, at least or about 90%, at least or about 95%, at least or about 98%, at least or about 99%, or 100% identity to those amino acids outside the interacting face of the peptide. Alternatively or in addition, amino acids outside those in the interacting face can include amino acid substitutions and/or deletions, whether conservative or not. For example, amino acids outside those in the interacting face can include 1, 2, 3, 4, 5, 6, 7, 8, less than 10, less than 5, less than 4, less than 3, or less than 2 amino acid substitutions, deletions, and/or additions, whether conservative or not.

In some embodiments, the ICL PTAIBs can be related to or can comprise features present in one or more of the (non-stapled) peptides disclosed in Pazgier et al., PNAS, 106; 4665-4670 (2009), which is hereby incorporated by reference in its entirety.

In some embodiments, the PTAIBs are internally cross-linked (ICL) (e.g., stapled or stitched) by one or more intra-peptide cross-linkers. “Peptide stapling” is a term coined from a synthetic methodology wherein two olefin-containing side-chains (e.g., cross-linkable side chains) present in a polypeptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). As used herein, the term “peptide stapling” includes the joining of two double bond-containing side-chains, two triple bond-containing side-chains, or one double bond-containing and one triple bond-containing side chain, which may be present in a polypeptide chain, using any number of reaction conditions and/or catalysts to facilitate such a reaction, to provide a singly “stapled” polypeptide. Additionally, the term “peptide stitching,” as used herein, refers to multiple and tandem (e.g., a single amino acid is cross-linked to two amino acids) “stapling” events in a single polypeptide chain to provide a “stitched” (multiply stapled) polypeptide. Peptide stitching is described in, e.g., WO 2008121767 and WO 2010/068684, which are both hereby incorporated by reference.

Stapling of a peptide using all-hydrocarbon cross-link has been shown to help maintain its native conformation and/or secondary structure, particularly under physiologically relevant conditions (see, e.g., Schafmiester et al., J. Am. Chem. Soc., 122:5891-5892, 2000; Walensky et al., Science, 305:1466-1470, 2004).

Stapling the PTAIBs herein by an all-hydrocarbon crosslink predisposed to have an alpha-helical secondary structure can constrain the PTAIB to its native alpha-helical conformation. The constrained secondary structure may, for example, increase the peptide's resistance to proteolytic cleavage, may increase the peptide's hydrophobicity, may allow for better penetration of the peptide into the target cell's membrane (e.g., through an energy-dependent transport mechanism such as pinocytosis), and/or may lead to an improvement in the peptide's biological activity relative to the corresponding non cross-linked (e.g., “unstitched” or “unstapled”) peptide. Such constraints have been applied to the apoptosis-inducing BID-BH3 alpha-helix, resulting in a higher suppression of malignant growth of leukemia in an animal model compared to the unstitched polypeptide (see, e.g., Walensky et al., Science, 305:1466-1470, 2004; U.S. 2005/02506890; and U.S. 2006/0008848, each of which is incorporated herein by reference). Suitable cross-links (e.g., which are also referred to in the art as tethers) are described herein and in, e.g., U.S. Patent Publication No. 2005/0250680, PCT/U52008/058575, USSN 12/864,375 (WO 2009/108261), and WO 2010/148335.

Cross-linked peptides disclosed herein can include natural and non-natural amino acids and have a linkage between the alpha carbons of two amino acids (replacing the side chain of those amino acids). Methods suitable for obtaining (e.g., synthesizing), stapling, and purifying the peptides disclosed herein are known in the art (see, e.g., Bird et. al., Methods in Enzymol., 446:369-386 (2008); Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety) and are described herein.

In some embodiments, such internally cross-linked (ICL) p53 peptides (PTAIBs) can exhibit a higher affinity for HDM2 and/or HDMX than a non-cross-linked or control peptide, e.g., a non-cross-linked peptide having the same amino acid sequence. In some embodiments, ICL PTAIBs can penetrate a cell membrane or have higher cell penetrability than a non-cross-linked or control peptide, e.g., a non-cross-linked peptide having the same amino acid sequence.

SEQ ID NO: 1 is the sequence of human p53, specifically:

(SEQ ID NO: 1)
Met Glu Glu Pro Gln Ser Asp Pro Ser Val Glu Pro
Pro Leu Ser Gln Glu Thr Phe Ser Asp Leu Trp Lys
Leu Leu Pro Glu Asn Asn Val Leu Ser Pro Leu Pro
Ser Gln Ala Met Asp Asp Leu Met Leu Ser Pro Asp
Asp Ile Glu Gln Trp Phe Thr Glu Asp Pro Gly Pro
Asp Glu Ala Pro Arg Met Pro Glu Ala Ala Pro Arg
Val Ala Pro Ala Pro Ala Ala Pro Thr Pro Ala Ala
Pro Ala Pro Ala Pro Ser Trp Pro Leu Ser Ser Ser
Val Pro Ser Gln Lys Thr Tyr Gln Gly Ser Tyr Gly
Phe Arg Leu Gly Phe Leu His Ser Gly Thr Ala Lys
Ser Val Thr Cys Thr Tyr Ser Pro Ala Leu Asn Lys
Met Phe Cys Gln Leu Ala Lys Thr Cys Pro Val Gln
Leu Trp Val Asp Ser Thr Pro Pro Pro Gly Thr Arg
Val Arg Ala Met Ala Ile Tyr Lys Gln Ser Gln His
Met Thr Glu Val Val Arg Arg Cys Pro His His Glu
Arg Cys Ser Asp Ser Asp Gly Leu Ala Pro Pro Gln
His Leu Ile Arg Val Glu Gly Asn Leu Arg Val Glu
Tyr Leu Asp Asp Arg Asn Thr Phe Arg His Ser Val
Val Val Pro Tyr Glu Pro Pro Glu Val Gly Ser Asp
Cys Thr Thr Ile His Tyr Asn Tyr Met Cys Asn Ser
Ser Cys Met Gly Gly Met Asn Arg Arg Pro Ile Leu
Thr Ile Ile Thr Leu Glu Asp Ser Ser Gly Asn Leu
Leu Gly Arg Asn Ser Phe Glu Val Arg Val Cys Ala
Cys Pro Gly Arg Asp Arg Arg Thr Glu Glu Glu Asn
Leu Arg Lys Lys Gly Glu Pro His His Glu Leu Pro
Pro Gly Ser Thr Lys Arg Ala Leu Pro Asn Asn Thr
Ser Ser Ser Pro Gln Pro Lys Lys Lys Pro Leu Asp
Gly Glu Tyr Phe Thr Leu Gln Ile Arg Gly Arg Glu
Arg Phe Glu Met Phe Arg Glu Leu Asn Glu Ala Leu
Glu Leu Lys Asp Ala Gln Ala Gly Lys Glu Pro Gly
Gly Ser Arg Ala His Ser Ser His Leu Lys Ser Lys
Lys Gly Gln Ser Thr Ser Arg His Lys Lys Leu Met
Phe Lys Thr Glu Gly Pro Asp Ser Asp

In some instances, PTAIBs can include the sequence Leu Ser Gln Glu Thr Phe Ser Asp Leu Trp Lys Leu Leu Pro Glu Asn (amino acids 14 to 29 of SEQ ID NO: 1 (SEQ ID NO: 2)). In any of the sequences, the side chains of two amino acids separated by 2, 3, 4, or 6 amino acids can be replaced by the linking group R₃.

In the stapled peptides, any position occupied by Gln can be Glu instead and any position occupied by Glu can be Gln instead. Similarly, any position occupied by Asn can be Asp instead and any position occupied by Aps can be Asn instead. The choice of Asn or Arg and Gln or Glu will depend on the desired charge of the stapled peptide.

A tether or cross-link can extend across the length of one or two helical turns (i.e., about 3.4 or about 7 amino acids). Accordingly, amino acids positioned at i and i+3; i and i+4; or i and i+7 are ideal candidates for chemical modification and cross-linking. Thus, for example, where a peptide has the sequence . . . Xaa₁, Xaa₂, Xaa₃, Xaa₄, Xaa₅, Xaa₆, Xaa₇, Xaa₈, Xaa₉ . . . (wherein, “. . . ” indicates the optional presence of additional amino acids), cross-links between Xaa₁ and Xaa₄, (e.g., 1+3) or between Xaa₁ and Xaa₅ (e.g., 1+4), or between Xaa₁ and Xaa₈ (e.g., i+7) are useful as are cross-links between Xaa₂ and Xaa₅ (e.g., i+3), or between Xaa₂ and Xaa₆ (e.g., i+4), or between Xaa₂ and Xaa₉ (e.g., i+7), etc. The polypeptides can include more than one crosslink within the polypeptide sequence to either further stabilize the sequence or facilitate the stabilization of longer polypeptide stretches. If the polypeptides are too long to be readily synthesized in one part, independently synthesized, ICL PTAIBs can be conjoined by a technique called native chemical ligation (see, e.g., Bang et al., J. Am. Chem Soc. 126:1377).

Alternatively or in addition, ICL PTAIBs can include one or more (e.g., one, two, three, four, five, six, seven, eight, nine, ten, or more, less than 10, less than 9, less than 8, less than 7, less than 6, less than 5, less than 4, less than 3, or less than 2 staples and/or stiches.

Internal cross-links (e.g., staples and/or stitches) can be positioned on amino acids within a peptide to conserve the structural relationship of amino acids in the binding or interacting face of the peptide (e.g., to preserve the binding interface of a peptide). For example, one or more of can be stapled or stitched to at least one other amino acid to conserve the structural relationship of amino acids in the hydrophobic interaction face (see, e.g., Kussie et al., Science, 274(5289):948-953 (1996), and Joseph et al., Cell Cycle, 9(22):4560-4568 (2010)). Such internal cross-links can include: one or more staples; one or more stitches; and/or a combination of one or more staples with one or more stitches. As noted above, exemplary ICL PTAIBs include, e.g., SAH-p53-8 (SEQ ID NO: 3).

Selection of amino acids for modification (e.g., to support an internal cross-link) can also be facilitated by staple scanning. The term “staple scan” refers to the synthesis of a library of stapled peptides whereby the location of the i and 1+3; i and 1+4; and i and i+7 single and multiple staple, or stitches, are positioned sequentially down the length of the peptide sequence, sampling all possible positions, to identify desired or optimal properties and activities for the stapled or stitched constructs.

In some instances, ICL PTAIBs include at least two internally cross-linked or stapled amino acids, wherein the at least two amino acids are separated by 2 (i.e., i, 1+3), 3 (i.e., i, 1+4), or 6 (i.e., i , i+7) amino acids. While at least two amino acids are required to support an internal cross-link (e.g., a staple), additional pairs of internally cross-linked amino acids can be included in a peptide, e.g., to support additional internal cross-links (e.g., staples). For example, peptides can include 1, 2, 3, 4, 5, or more staples.

Alternatively, or in addition, ICL PTAIBs can include three internally cross-linked or stitched amino acids. A peptide stitch includes at least three internally cross-linked amino acids, wherein the middle of the three amino acids (referred to here as the core or central amino acid) forms an internal cross-link (between alpha carbons) with each of the two flanking modified amino acids. The core amino acid includes two internally cross-linked side chains, which can be saturated or not saturated. Amino acids cross-linked to the core amino acid can be separated from the core amino acid in either direction by 2, 3, or 6 amino acids (e.g., i, i−3, i, i−4, i, i−7, i, i+3, i, i+4, i, i+7, where “i” is the core amino acid). The number of amino acids on either side of the core (e.g., between the core amino acid and an amino acid cross-linked to the core) can be the same or different. In some instances, a stitch can include 3, 4, 5, or more internally cross-linked amino acids. In some instances, peptides can include 1, 2, 3, 4, 5, or more stitches.

In some embodiments, peptides herein can include a combination of at least one (e.g., 1, 2, 3, 4, or 5) staple and at least one (e.g., 1, 2, 3, 4, or 5) stitch.

In some embodiments, the tethers, e.g., hydrocarbon staples are used to stabilize structures other than helices. In such cases, the ends of the tethers can be placed at intervals other than at i, i+3, i+4, and i+7.

As disclosed above, peptides herein include at least two modified amino acids that together form an internal (intramolecular) cross-link, wherein the at least two modified amino acids are separated by 2 (i.e., i, i+3), 3 (i.e., i, 1+4), or 6 (i.e., i , i+7) amino acids.

The peptides may contain one or more asymmetric centers and thus occur as racemates and racemic mixtures, single enantiomers, individual diastereomers and diastereomeric mixtures and geometric isomers (e.g., Z or cis and E or trans) of any olefins present. All such isomeric forms of these compounds are expressly included in the present invention. The compounds may also be represented in multiple tautomeric forms, in such instances, the invention expressly includes all tautomeric forms of the compounds described herein (e.g., isomers in equilibrium (e.g., keto-enol), wherein alkylation at multiple sites can yield regioisomers), regioisomers, and oxidation products of the compounds disclosed herein (the invention expressly includes all such reaction products). All such isomeric forms of such compounds are included as are all crystal forms.

The peptides can also include amino acids containing both an amino group and a carboxyl group bonded to a carbon referred to as the alpha carbon. Also bonded to the alpha carbon is a hydrogen and a side-chain. Suitable amino acids include, without limitation, both the D- and L-isomers of the 20 common naturally occurring amino acids found in peptides (e.g., A, R, N, C, D, Q, E, G, H, I, L, K, M, F, P, S, T, W, Y, V (as known by their one-letter abbreviations)) as well as the naturally occurring and unnaturally occurring amino acids prepared by organic synthesis or other metabolic routes.

### Modification of Hydrocarbon Tethers

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation or dihydroxylation) to provide one of compounds below.

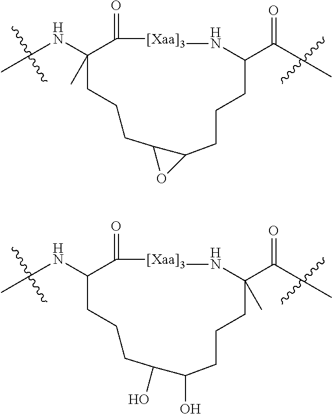

Either the epoxide moiety or one of the free hydroxyl moieties can be further functionalized. For example, the epoxide can be treated with a nucleophile, which provides additional functionality that can be used, for example, to attach a tag (e.g., a radioisotope or fluorescent tag). The tag can be used to help direct the compound to a desired location in the body or track the location of the compound in the body. Alternatively, an additional therapeutic agent can be chemically attached to the functionalized tether (e.g., an anti-cancer agent such as rapamycin, vinblastine, taxol, etc.). Such derivatization can alternatively be achieved by synthetic manipulation of the amino or carboxy terminus of the polypeptide or via the amino acid side chain. Other agents can be attached to the functionalized tether, e.g., an agent that facilitates entry of the polypeptide into cells.

While hydrocarbon tethers have been described, other tethers are also envisioned. For example, the tether can include one or more of an ether, thioether, ester, amine, or amide moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid.

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while examples of tethers spanning from amino acids i to i+3, i to 1+4, and i to i+7 have been described in order to provide a tether that is primarily on a single face of the alpha helix, the tethers can be synthesized to span any combinations of numbers of amino acids.

It is further envisioned that the staple itself may contribute to binding interactions at the surface of the target protein binding site, and thus, may be used to increase affinity while retaining target affinity, as has been reported (Stewart et al, Nature Chem. Biol., 2010; Joseph et al, Cell Cycle, 2010 (supra)).

In some instances, alpha disubstituted amino acids are used in the polypeptide to improve the stability of the alpha helical secondary structure. However, alpha disubstituted amino acids are not required, and instances using mono-alpha substituents (e.g., in the tethered amino acids) are also envisioned.

In some instances, it can be useful to create an inactive stapled peptide by replacing one or more (e.g., all three) of Phe6, Trpio, Leui3 of the interacting face of p53 (e.g., of SEQ ID NO: 1) with another amino acid, e.g., Ala. Such inactive stapled peptides can be useful, for example, as negative controls.

The stapled polypeptides can include a drug, a toxin, a derivative of polyethylene glycol; a second polypeptide; a carbohydrate, etc. Where a polymer or other agent is linked to the stapled polypeptide is can be desirable for the composition to be substantially homogeneous.

The addition of polyethylene glycol (PEG) molecules can improve the pharmacokinetic and pharmacodynamic properties of the polypeptide. For example, PEGylation can reduce renal clearance and can result in a more stable plasma concentration. PEG is a water soluble polymer and can be represented as linked to the polypeptide as formula:

XO—(CH₂CH₂O)ₙ—CH₂CH₂—Y

where n is 2 to 10,000 and Xis H or a terminal modification, e.g., a C₁₋₄ alkyl; and Y is an amide, carbamate or urea linkage to an amine group (including but not limited to, the epsilon amine of lysine or the N-terminus) of the polypeptide. Y may also be a maleimide linkage to a thiol group (including but not limited to, the thiol group of cysteine). Other methods for linking PEG to a polypeptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in, e.g., WO 99/34833; WO 99/14259, and U.S. Pat. No. 6,348,558.

In certain embodiments, macromolecular polymer (e.g., PEG) is attached to an agent described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

The stapled peptides can also be modified, e.g., to facilitate cellular uptake or increase in vivo stability, in some embodiments. For example, acylating or PEGylating a peptidomimetic macrocycle facilitates cellular uptake, increases bioavailability, increases blood circulation, alters pharmacokinetics, decreases immunogenicity and/or decreases the needed frequency of administration.

In some embodiments, the ICL PTAIBs have an enhanced ability to penetrate cell membranes (e.g., relative to non-stapled peptides). These same ICL PTAIBs can also possess, or can be modified to possess, an apparent affinity to human serum proteins of 1 μM or weaker. In another embodiment, the improved ICL PTAIB possesses an apparent affinity to human serum proteins of 3 μM or weaker. In another embodiment, the improved ICL PTAIB possesses an apparent affinity to human serum proteins of 10 μM or weaker. In another embodiment, the improved ICL PTAIB possesses an apparent affinity to human serum proteins of 70 μM or weaker. In another embodiment, the improved ICL PTAIB possesses an apparent affinity to human serum proteins of between 1-70 μM. In another embodiment, the improved ICL PTAIB possesses an apparent affinity to human serum proteins of between 1-700 μM. In some embodiments, the improved ICL PTAIB possesses an estimated free fraction in whole blood of between 0.1-50%. In another embodiment, the improved ICL PTAIB possesses an estimated free fraction in whole blood of between 0.5-10%. For example, a polypeptide can be selected such that the apparent serum binding affinity (Kd*) of the crosslinked polypeptide is 1, 3, 10, 70 μM or greater. In other embodiments, the Kd* of the crosslinked polypeptide is 1 to 10, 70, or 700 μM. In other embodiments, the crosslinked polypeptides are selected such that it possesses an estimated free fraction in human blood of between 0.1 and 50%, or between 0.15 and 10%. Methods for quantifying the propensity for any particular peptide to bind to serum proteins are known in the art (see, e.g., U.S. Patent Application Publication No. 2010/0216688, published Aug. 26, 2010).

In some embodiments, the improved ICL PTAIB possesses an estimated free fraction in whole blood of between 0.1-50%. In another embodiment, the improved ICL PTAIB possesses an estimated free fraction in whole blood of between 0.5-10%.

### Methods of Synthesis

As noted above, methods of synthesizing the compounds of the described herein are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, e.g., those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The peptides of this invention can be made by chemical synthesis methods, which are well known to the ordinarily skilled artisan. See, e.g., Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH₂ protected by either t-Boc or Fmoc chemistry using side chain protected amino acids on, e.g., an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from Advanced Chemtech.

In the modified polypeptides, one or more conventional peptide bonds replaced by a different bond that may increase the stability of the polypeptide in the body. Peptide bonds can be replaced by: a retro-inverso bonds (C(O)—NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The polypeptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. The polypeptides of the invention may also be conjugated to, for example, polyethylene glycol (PEG); alkyl groups (e.g., C1-C20 straight or branched alkyl groups); fatty acid radicals; and combinations thereof.

α,α-Disubstituted non-natural amino acids containing olefinic side chains of varying length can be synthesized by known methods (see, e.g., Williams et al. J. Am. Chem. Soc., 113:9276, 1991; Schafmeister et al., J. Am. Chem Soc., 122:5891, 2000; and Bird et al., Methods Enzymol., 446:369, 2008). For peptides where an i linked to i+7 staple is used (two turns of the helix stabilized) either one S amino acid and one R₈ are used, or one S₈ amino acid and one R₅ amino acid are used. R₈ is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin.

Fmoc-protected α-amino acids (other than the olefinic amino acids Fmoc-S₅—OH, Fmoc-R₈—OH, Fmoc-R₈—OH, Fmoc-S₈—OH and Fmoc-R₅—OH), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is known in the art (see, e.g., Williams et al., Org. Synth., 80:31, 2003).

In some embodiments, stapled peptides can be generated using the following method. Peptides can be synthesized manually using Fmoc solid phase peptide chemistry on Rink amide MBHA resin with loading levels of 0.4-0.6 mmol/g resin. The following protocol was used:
1. The Fmoc protective group was removed with 20% piperidine in NMP for 30 min.
2. The resin was washed with NMP five times.
3. The subsequent Fmoc-protected amino acid was coupled for 30 min (60 min for a cross-linker) using Fmoc-AA (10 equiv., 4 equiv. for a cross-linker), HCTU (9.9 equiv., 3.9 equiv. for a cross-linker), and DIEA (20 equiv., 7.8 equiv. for a cross-linker).
4. The resin was washed with NMP five times.
5. Repeat from step 1.

Peptides can be capped with, e.g., an Ac or a β-alanine residue at the N-terminus. CD experiments make use of peptides that have been acetylated at the N-terminus. The acetylation reaction consisted of deprotection of the Fmoc group as outlined above, followed by reaction with acetic anhydride and DIEA. All other experiments shown make use of fluoresceinated peptides at the N-terminus. To this end, the peptides with the deprotected N-terminus were exposed to fluorescein isothiocyanate in DMF overnight in the presence of DIEA.

Ring-closing metathesis reaction can be performed on the N-terminal capped peptides while still on the solid support in a disposable fritted reaction vessel. The resin was exposed to a 10 mM solution of bis(tricyclohexylphosphine)benzylidine ruthenium (IV) dichloride (Grubbs First Generation Catalyst) in 1,2-dichloroethane or dichloromethane for 2 hours. The catalyst addition and 2 hour metathesis reaction was repeated once. The resin-bound peptide was washed with CH₂Cl₂ three times and dried under a stream of nitrogen.

Peptides can be cleaved from the resin and deprotected by exposure to Reagent K (82.5% TFA, 5% thioanisole, 5% phenol, 5% water, 2.5% 1,2-ethanedithiol) or 95% TFA, 2.5% water, 2.5% triisopropylsilane and precipitated with methyl-tent-butyl ether at 4° C. and lyophilized. Peptides can be purified, e.g., using HPLC and optionally lyophilized.

In some embodiments, the peptides are substantially free of non-stapled peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50%, or 60% DMSO. In a specific embodiment, a 50%/50% DMSO/NMP solution is used. The solution may be incubated for a period of 1, 6, 12 or 24 hours, following which the resin may be washed, for example with dichloromethane or NMP. In one embodiment, the resin is washed with NMP. Shaking and bubbling an inert gas into the solution may be performed.

### Assays

Properties of the ICL PTAIBs can be assayed, for example, using the methods described below.

Assays to Determine Alpha-Helicity: The ICL PTAIBs are dissolved in an aqueous solution (e.g. 50 mM potassium phosphate solution at pH 7, or distilled H₂O, to concentrations of 25-50 μM). Circular dichroism (CD) spectra are obtained on a spectropolarimeter (e.g., Jasco J-710) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The cc-helical content of each peptide is calculated by dividing the mean residue ellipticity by the reported value for a model helical decapeptide (see, e.g., Yang et al., Methods Enzymol. 130:208 (1986)).

Assays to Determine Melting Temperature (Tm): ICL PTAIBs or unmodified peptides are dissolved in distilled H₂O (e.g. at a final concentration of 50 μM) and Tm is determined by measuring the change in ellipticity over a temperature range (e.g. 4 to 95° C.) on a spectropolarimeter (e.g., Jasco J-710) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

Protease Resistance Assays: The amide bond of the peptide backbone is susceptible to hydrolysis by proteases, thereby rendering peptidic compounds vulnerable to rapid degradation in vivo. Peptide helix formation, however, typically buries the amide backbone and therefore may shield it from proteolytic cleavage. The ICL PTAIBs may be subjected to in vitro trypsin proteolysis to assess for any change in degradation rate compared to a corresponding un-cross-linked polypeptide. For example, the ICL PTAIB and a corresponding un-cross-linked polypeptide are incubated with trypsin agarose and the reactions quenched at various time points by centrifugation and subsequent HPLC injection to quantitate the residual substrate by ultraviolet absorption at 280 nm. Briefly, the ICL PTAIB and unmodified precursor (5 mcg) are incubated with trypsin agarose (Pierce) (enzyme to substrate (E/S) ratio of, e.g., about 1:100 or about 1:125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time.

Ex Vivo Stability Assays: ICL PTAIBs and/or a corresponding un-cross-linked polypeptide can be each incubated with fresh mouse, rat and/or human serum (e.g. 1-2 mL) at 37° C. for, e.g., 0, 1, 2, 4, 8, and 24 hours. Samples of differing macrocycle concentration may be prepared by serial dilution with serum. To determine the level of intact compound, the following procedure may be used: The samples are extracted by transferring 100 μL of sera to 2 ml centrifuge tubes followed by the addition of 10 μL of 50% formic acid and 500 μL acetonitrile and centrifugation at 14,000 RPM for 10 min at about 4° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

In Vitro Binding Assays: To assess the binding and affinity of ICL PTAIBs and unmodified precursors to acceptor proteins, a fluorescence polarization assay (FPA) can be used, for example. The FPA technique measures the molecular orientation and mobility using polarized light and fluorescent tracer. When excited with polarized light, fluorescent tracers (e.g., FITC) attached to molecules with high apparent molecular weights (e.g., FITC-labeled peptides bound to a large protein) emit higher levels of polarized fluorescence due to their slower rates of rotation as compared to fluorescent tracers attached to smaller molecules (e.g., FITC-labeled peptides that are free in solution).

In Vitro Displacement Assays to Characterize Antagonists of Peptide-Protein Interactions: To assess the binding and affinity of compounds that antagonize the interaction between a peptide and an acceptor protein, a fluorescence polarization assay (FPA) utilizing a fluoresceinated ICL PTAIB derived from an unmodified precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g., FITC-labeled peptides bound to a large protein) emit higher levels of polarized fluorescence due to their slower rates of rotation as compared to fluorescent tracers attached to smaller molecules (e.g., FITC-labeled peptides that are free in solution). A compound that antagonizes the interaction between the fluoresceinated ICL PTAIB and an acceptor protein will be detected in a competitive binding FPA experiment.

Binding Assays in Intact Cells: It is possible to measure binding of peptides or ICL PTAIBs to their natural acceptors by immunoprecipitation experiments, e.g., as described herein, or by measuring protein interaction disruption directly in live/intact cells using a luciferase reconstitution system based on inducible formation of p53-MDM2 and/or p53-MDMX protein complexes (see, e.g., Li et al., Cell Rep 2014, 9: 1946-58).

P-LISA and Immunofluorescence. To assess the capacity of SAH-p53-8 to disrupt intracellular complexes of p53/HDMX in intact cells, a P-LISA assay was applied. U2OS cells expressing a doxycycline-inducible HA-HDMX construct (Wang et al., 2007) were seeded onto coverslips and treated with doxycycline for 24 h. SAH-p53-8 (10 μM), enantiomeric Nutlin-3 (10 μM) (Roche), or both compounds were added for the final 8 h of treatment. The cells were fixed in 3.7% paraformaldehyde, washed in PBS, and permeabilized in 0.2% Triton X-100 for 5 min. Coverslips were then blocked in 10% normal goat serum in PBS (NGS) for 2 h. For P-LISA, primary antibodies HA.11 (BabCo, 1:500) and FL393 (Santa Cruz, 1:1000) were diluted in PBS/EDTA/0.2% Triton X-100/2% NGS and incubated at 4° C. overnight. Following washes with TBS/0.05% TWEEN™ 20, a proximity ligation in situ assay (P-LISA) was performed according to the manufacturer's protocol (Detection Kit 613, OLink Bioscience) with the following exception: goat anti-rabbit (minus) and anti-mouse (plus) P-LISA probes were diluted in NGS at 1:10 instead of 1:5. Coverslips were mounted on microscope slides and images acquired using OpenLab software (Improvision) and a Zeiss Axioplan 2 microscope. Nuclear foci (at least 100 cells per treatment) were quantified using Blobfinder software (Centre for Image Analysis, Uppsala University, Sweden). All exposure times and intensity thresholds were set based on doxycycline/Nutlin-3 co-treatment and kept constant for each treatment. The statistical significance of the observed differences in foci number among the treatment conditions was determined using the unpaired t-test with Welch's correction. For standard immunofluorescence imaging of p53 and HDMX, the antibodies indicated above were again employed but following the PBS washes, the slides were incubated (1 h, room temperature) with goat anti-rabbit AF568 (1:1000) and goat anti-mouse AF488 (1:500) (Invitrogen/Molecular Probes) containing 1 μg/mL Hoechst. Density slices from each Hoechst image were generated in OpenLab, and used as masks to quantify the nuclear intensity of both p53 and HDMX. Total intensity was defined as average pixel intensity x nuclear area, and was corrected for nuclear size differences. Graphical representation and statistical analyses were performed using Microsoft Excel and Prism software (GraphPad).

Cellular Penetrability Assays: To measure the cell penetrability of peptides or crosslinked polypeptides, intact cells are incubated with fluoresceinated crosslinked polypeptides (10 μM) for 4 hrs in serum-free media or in media supplemented with human serum at 37° C., washed twice with media and incubated with trypsin (0.25%) for 10 min at 37° C. The cells are washed again and resuspended in PBS. Cellular fluorescence is analyzed, for example, by using either a FACSCalibur flow cytometer or Cellomics' KineticScan® HCS Reader. Alternative methods include, e.g., high content epifluorescence microscopy, confocal imaging, or fluorescence scan of electrophoresed lysates from FITC-peptide treated cells (see, e.g., LaBelle et al. JCI 2012, 122: 2018-31).

Cellular Efficacy Assays: The efficacy of certain ICL PTAIBs is determined, for example, in cell-based killing assays using a variety of tumorigenic and non-tumorigenic cell lines and primary cells derived from human or mouse cell populations. Cell viability is monitored, for example, over 24-96 hrs of incubation with crosslinked polypeptides (0.5 to 50 μM) to identify those that kill at EC50<10 μM. Several standard assays that measure cell viability are commercially available and are optionally used to assess the efficacy of the crosslinked polypeptides. In addition, assays that measure Annexin V and caspase activation are optionally used to assess whether the crosslinked polypeptides kill cells by activating the apoptotic machinery. For example, the Cell Titer-Glo™ assay is used which determines cell viability as a function of intracellular ATP concentration.

In Vivo Stability Assays: To investigate the in vivo stability of ICL PTAIBs, the compounds are, for example, administered to mice and/or rats by IV, IP, PO or inhalation routes at concentrations ranging from 0.1 to 50 mg/kg and blood specimens withdrawn at 0 min, 5 min, 15 min, 30 min, 1 h, 4 h, 8 h, and 24 h post-injection. Levels of intact compound in 25 viL of fresh serum are then measured by LC-MS/MS as above.

In Vivo Efficacy in Animal Models: To determine the anti-oncogenic activity of ICL PTAIBs in vivo, the compounds are, for example, given alone (IP, IV, PO, by inhalation or nasal routes) or in combination with sub-optimal doses of relevant chemotherapy (e.g., cyclophosphamide, doxorubicin, etoposide). Leukemia can be monitored, for example, by injecting mice with D-luciferin (60 mg/kg) and imaging the anesthetized animals (e.g., Xenogen In Vivo Imaging System, Caliper Life Sciences, Hopkinton, Mass.). Total body bioluminescence is quantified by integration of photonic flux (photons/sec) by Living Image Software (Caliper Life Sciences, Hopkinton, Mass.). ICL PTAIBs alone or in combination with sub-optimal doses of relevant chemotherapeutics agents are, for example, administered to leukemic mice (10 days after injection/day 1 of experiment, in bioluminescence range of 14-16) by tail vein or IP routes at doses ranging from 0.1 mg/kg to 50 mg/kg for 7 to 21 days. Optionally, the mice are imaged throughout the experiment every other day and survival monitored daily for the duration of the experiment. Expired mice are optionally subjected to necropsy at the end of the experiment. Another animal model is implantation into NOD-SCID mice of DoHH2, a cell line derived from human follicular lymphoma that stably expresses luciferase. Another animal model is implantation into NOD-SCID-IL2Rγnull (NSG) mice of Luc-JEG-3, a cell line derived from human choriocarcinoma that stably expresses luciferase. These in vivo tests can optionally generate preliminary pharmacokinetic, pharmacodynamics, and/or toxicology data.

Clinical Trials: To determine the suitability of the crosslinked polypeptides of the invention for treatment of humans, clinical trials can be performed. For example, patients diagnosed with cancer and in need of treatment are selected and separated in treatment and one or more control groups, wherein the treatment group is administered an ICL PTAIB, while the control groups receive a placebo or a known anti-cancer drug. The treatment safety and efficacy of the ICL PTAIBs can thus be evaluated by performing comparisons of the patient groups with respect to factors such as survival and quality-of-life. In this example, the patient group treated with an ICL PTAIB show improved long-term survival compared to a patient control group treated with a placebo.

### Compositions

Any combination of the PTAIBs disclosed herein can be used or administered in combination with one or more other compositions and/or methods for inducing p53 expression and/or activity, and/or activating cell death pathways through other means. Exemplary compositions and/or methods for inducing p53 expression and/or activity can include, but are not limited to, e.g., ionizing radiation, ultraviolet light, and/or DNA damaging agents (e.g., etoposide, actinomycin D, doxorubicin, paclitaxel, and/or other chemotherapeutic agents).

In some embodiments, p53 activity in a cell can be increased by introducing active p53 into a cell (e.g., using viruses (e.g., retroviruses) and/or DNA transduction). In some embodiments, the active p53 can be expressed from a nucleic acid sequence obtained from the subject and/or the active p53 can be an isolated protein obtained from the subject and optionally coupled to a moiety that increases cell penetrability of the p53. In some embodiments, p53 activity can be increased by retroviral reconstruction of p53 in a targeted fashion in cancer cells (e.g., cancer cells with diminished p53 activity).

As used herein, the term “expression” includes protein and/or nucleic acid expression and/or protein activity.

As used herein, the ICL PTAIBs, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound or agent disclosed herein which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The ICL PTAIBs may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, pamoate, phosphate, picrate, pivalate, propionate, salicylate, succinate, sulfate, tartrate, tosylate, trifluoromethylsulfonate, and undecanoate. Salts derived from appropriate bases include alkali metal (e.g., sodium), alkaline earth metal (e.g., magnesium), ammonium and N-(alkyl)4+ salts. This invention also envisions the quaternization of any basic nitrogen-containing groups of the compounds disclosed herein. Water or oil-soluble or dispersible products may be obtained by such quaternization.

The ICL PTAIBs described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.001 to about 100 mg/kg of body weight, or according to the requirements of the particular drug. Alternatively, or in addition, the present invention may be administered according to any of the Food and Drug Administration approved methods, for example, as described in the FDA Data Standards Manual (DSM) (available at

<http://www.fda.gov/Drugs/DevelopmentApprovalProcess/FormsSubmissionRequirements/ElectronicSubmissions/DataStandardsManualmonographs>).

The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

In some embodiments, an effective dose of an ICL PTAIB can include, but is not limited to, e.g., about, 0.00001, 0.0001, 0.001, 0.01, 0.1, 1 or 10-10000; 0.00001, 0.0001, 0.001, 0.01, 0.1, 1 or 10-5000; 0.00001, 0.0001, 0.001, 0.01, 0.1, 1 or 10-2500; 0.00001, 0.0001, 0.001, 0.01, 0.1, 1 or 10-1000; 0.00001, 0.0001, 0.001, 0.01, 0.1, 1 or 10-900; 0.00001, 0.0001, 0.001, 0.01, 0.1, 1 or 10-800; 0.00001, 0.0001, 0.001, 0.01, 0.1, 1 or 10-700; 0.00001, 0.0001, 0.001, 0.01, 0.1, 1 or 10-600; 0.00001, 0.0001, 0.001, 0.01, 0.1, 1 or 10-500; 0.00001, 0.0001, 0.001, 0.01, 0.1, 1 or 10-400; 0.00001, 0.0001, 0.001, 0.01, 0.1, 1 or 10-300; 0.00001, 0.0001, 0.001, 0.01, 0.1, 1 or 10-200; 0.00001, 0.0001, 0.001, 0.01, 0.1, 1 or 10-100; 0.00001, 0.0001, 0.001, 0.01, 0.1, 1 or 10-90; 0.00001, 0.0001, 0.001, 0.01, 0.1, 1 or 10-80; 0.00001, 0.0001, 0.001, 0.01, 0.1, 1 or 10-70; 0.00001, 0.0001, 0.001, 0.01, 0.1, 1 or 10-60; 0.00001, 0.0001, 0.001, 0.01, 0.1, 1 or 10-50; 0.00001, 0.0001, 0.001, 0.01, 0.1, 1 or 10-40; 0.00001, 0.0001, 0.001, 0.01, 0.1, 1 or 10-30; 0.00001, 0.0001, 0.001, 0.01, 0.1, 1 or 10-20; 0.00001, 0.0001, 0.001, 0.01, 0.1, 1 or 10-30; 0.00001, 0.0001, 0.001, 0.01, 0.1, 1-15, 0.00001, 0.0001, 0.001, 0.01, 0.1, 1 or 10-30; 0.00001, 0.0001, 0.001, 0.01, 0.1, 1 -10, 0.00001, 0.0001, 0.001, 0.01, 0.1, 1 or 10-30; or 0.00001, 0.0001, 0.001, 0.01, 0.1, 1-5 mg/kg/day, e.g., administered intravenously.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

Pharmaceutical compositions of this document comprise an ICL PTAIB or a pharmaceutically acceptable salt thereof; an additional agent including for example, morphine or codeine; and any pharmaceutically acceptable carrier, adjuvant or vehicle. Alternate compositions of this invention comprise a compound of the formulae described herein or a pharmaceutically acceptable salt thereof; and a pharmaceutically acceptable carrier, adjuvant or vehicle. The compositions delineated herein include the compounds of the formulae delineated herein, as well as additional therapeutic agents if present, in amounts effective for achieving a modulation of disease or disease symptoms.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as TWEENS™ or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intra-articular, intraarterial, intrasynovial, intrasternal, intrathecal, intralesional, and intracranial injection or infusion techniques. Alternatively or in addition, the present invention may be administered according to any of the Food and Drug Administration approved methods (as described above).

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, e.g., TWEEN™ 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as, e.g., olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and/or suspensions. Other commonly used surfactants such as TWEENS™, SPANS™, and/or other similar emulsifying agents or bioavailability enhancers which are commonly used in the manufacture of pharmaceutically acceptable solid, liquid, or other dosage forms may also be used for the purposes of formulation.

The pharmaceutical compositions of this document may be orally administered in any orally acceptable dosage form including, but not limited to, capsules, tablets, emulsions and aqueous suspensions, dispersions and solutions. In the case of tablets for oral use, carriers which are commonly used include, e.g., lactose and corn starch. Lubricating agents, such as, e.g., magnesium stearate, are also typically added. For oral administration in a capsule form, useful diluents include, e.g., lactose and dried corn starch. When aqueous suspensions and/or emulsions are administered orally, the active ingredient may be suspended or dissolved in an oily phase is combined with emulsifying and/or suspending agents. If desired, certain sweetening agents, flavoring agents, and/or coloring agents may be added.

The pharmaceutical compositions of this document may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, e.g., cocoa butter, beeswax, and polyethylene glycols.

The pharmaceutical compositions of this document may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this document comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, e.g., as part of a multiple dose regimen, from the compounds of this invention. Alternatively, those agents may be part of a single dosage form, mixed together with the compounds of this invention in a single composition.

Effective amounts of one or more compounds or a pharmaceutical composition for use in the present invention include amounts that promote increased p53 levels (e.g., protein levels) and/or p53 activity (e.g., biological activity) in a cell. A therapeutically effective amount of a compound is not required to cure a disease but will provide a treatment for a disease.

In some embodiments, the present disclosure provides methods for using any one or more of the compositions (indicated below as ‘X’) disclosed herein in the following methods:

Substance X for use as a medicament in the treatment of one or more diseases or conditions disclosed herein (e.g., cancer, referred to in the following examples as ‘Y’). Use of substance X for the manufacture of a medicament for the treatment of Y; and substance X for use in the treatment of Y.

### Assays and Methods of Treatment

We have developed an assay and method of treatment for optimizing the use of ALRN-7041 and/or one or more other ICL PTAIBs in treating pediatric cancers. Genetic pressure to mutate p53, a common feature of human cancers, is mitigated in cancers overexpressing HDM2 and/or HDMX (e.g., pediatric myeloid leukemias). Thus, an assay assessing the status of wild-type and/or functional p53 in a pediatric cancer patient in the context of genetic amplification or overexpression of HDM2 and/or HDMX can be used as a biomarker (i.e., a “signature”) for predicting the efficacy of treating the patient with ALRN-7041 and/or one or more other ICL PTAIBs. The assay can be used to rapidly select patients for treatment with ALRN-7041 and/or one or more other ICL PTAIBs, and/or to optimize the administration of ALRN-7041 and/or one or more other ICL PTAIBs to a patient. For example, a cancer patient with generally wild-type and/or functional p53 coupled with overexpression of HDM2 and/or HDMX could be treated with ALRN-7041 and/or one or more other ICL PTAIBs, compared to a different patient with little to no wild-type and/or functional p53 or little or no expression of HDM2 and/or HDMX, who would have little to no response to ALRN-7041 and/or one or more other ICL PTAIBs. Thus, this disclosure provides a new therapeutic strategy for treating pediatric cancers such as AML, based on reactivating one of the most potent tumor suppressor proteins in all of human cancer.

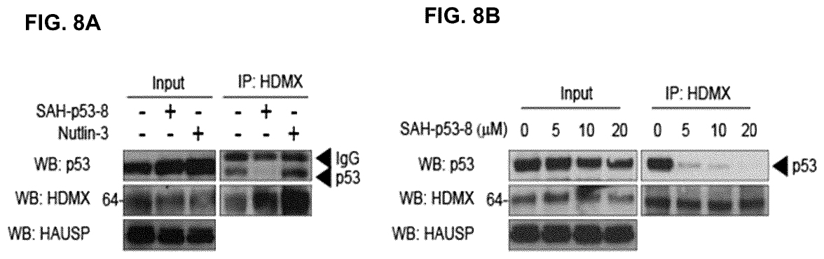

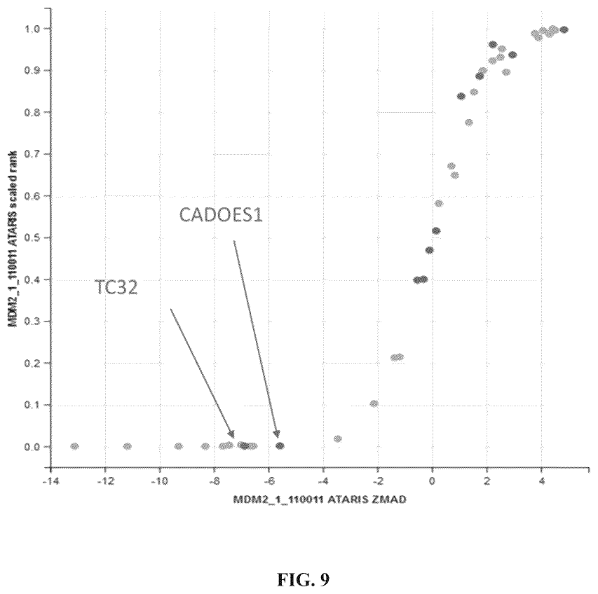

Alternately or in addition, an assay can assess the relative levels of suppressive p53/HDM2 and/or p53/HDMX complexes. Similar to the assay described above, an assay assessing the relative levels of p53/HDM2 and/or p53/HDMX complexes can be used to predict the efficacy of treating a patient with ALRN-7041 and/or one or more other ICL PTAIBs. The assay can be used to rapidly select patients for treatment with ALRN-7041 and/or one or more other ICL PTAIBs, and/or to optimize the administration of ALRN-7041 and/or one or more other ICL PTAIBs to a patient. As a proof of concept, we have used the assay to demonstrate the unique capacity of SAH-p53-8, but not Nutlin-3, to dissociate the inhibitory p53/HDMX complex in solid tumor cells (FIG. 8).

For example, to measure the levels of p53, HDM2, and HDMX, a plate (e.g., a 96-well polystyrene strip microplate (Corning 2592)) is coated with one or more capture antibodies specific to p53 (e.g., 15A5 rabbit monoclonal or PAb240 mouse monoclonal), HDM2 (e.g., 1A7 clone), and/or HDMX (e.g., MDMX-82 clone). After incubation (e.g., overnight at 4° C.), the plate is washed and blocked (e.g., with 1% BSA in PBS). The plate is then subjected to a sequence of serial washes, incubation with leukemia cell lysate (e.g., obtained from a cancer patient), serial washes, and detection using antibodies directed against p53 (e.g., DO-1 or 1C12 mouse monoclonal or FL-393 rabbit polyclonal depending on the species of the capture antibody), HDM2 (e.g., N-20 or IF-2 mouse monoclonal or 2A10 rabbit polyclonal), and/or HDMX (e.g., Bethyl-1258 rabbit polyclonal), respectively. The plates are developed using a secondary antibody conjugated to, e.g., horseradish peroxidase (HRP), followed by exposure to, e.g., a chromogenic HRP substrate (e.g., tetramethylbenzidine). After addition of a stop solution (e.g., 0.16 M sulfuric acid), the plate is analyzed by a reader (e.g., a Spectramax M5 microplate reader). For example, if the plates are developed using a secondary antibody conjugated to HRP and exposed to a chromogenic HRP substrate, the plate is analyzed by a reader with an absorbance setting of 450 nm. The concentration of each protein can be determined by correlation to a calibration curve using recombinant p53, HDM2, and/or HDMX protein standard solutions.

For example, to assess the relative levels of suppressive p53 complexes (i.e., p53/HDM2 and/or p53/HDMX) in pediatric leukemia cell samples (e.g., obtained from a pediatric cancer patient), plates coated with p53 capture antibody are again employed. After treatment of the blocked plate with leukemia cell lysate samples, the wells are treated with detection antibodies specific to HDM2 (e.g., N-20 or IF-2 mouse monoclonal or 2A10 rabbit polyclonal) or HDMX (e.g., Bethyl-1258 rabbit polyclonal). As above, the plate is then treated with secondary antibody followed by a chromogenic substrate and then absorbance measured at an appropriate wavelength (e.g., 450 nm). The concentration of HDM2 and HDMX detected in the anti-p53 plates reflects the levels of p53/HDM2 and p53/HDMX complexes as quantified by comparison to the recombinant HDM2 and HDMX calibration curves. Alternately or in addition, leukemia cell samples are exposed to a serial dilution (starting from, e.g., a concentration of 20 μM) of ALRN-7041, its mutant controls, and/or Nutlin-3a (a selective HDM2 inhibitor) for, e.g., 6 hours in the appropriate culture media, followed by the preparation of lysates for quantitation of p53/HDM2 and/or p53/HDMX complexes, performed as described above. Thus, we can determine whether a particular cancer presents an optimal biochemical set up for p53 reactivation by dual HDM2/HDMX inhibition (e.g., with ALRN-7041), and can also determine and/or optimize the amount/concentration of ALRN-7041 and/or one or more other ICL PTAIBs needed to effectively treat the cancer.

In any or all of the assays described herein, a mutant control peptide, e.g., ALRN-7342 (F19A), can optionally be used to confirm the effect of ALRN-7041 and/or one or more other ICL PTAIBs. ALRN-7342 (F19A) is identical to ALRN-7041 except for a single amino acid substitution (i.e., F19A), which destroys the ability of the peptide to bind to HDM2 and/or HDMX.

The assays described herein, alone or in combination, can be used to identify pediatric cancers and/or types of pediatric cancers generally susceptible to, or likely to be susceptible to, treatment with ALRN-7041 and/or one or more other ICL PTAIBs. For example, we have discovered that a large subset of pediatric cancers are unexpectedly susceptible to treatment with these peptides. These include, but are not limited to, e.g., the following pediatric cancers, including their presenting, relapsed, and/or refractory subtypes: acute myeloid leukemia (AML), acute lymphoblastic leukemia (ALL) (including T cell lineage ALL and B cell lineage ALL), Ewing sarcoma, retinoblastoma, neuroblastoma, glioma (including, e.g., diffuse interstitial pontine glioma (DIPG)), medulloblastoma, rhabdomyosarcoma (including, e.g., alveolar rhabdomyosarcoma and embryonal rhabdomyosarcoma), Wilm's tumor, and malignant rhabdoid tumor (MRT). The compounds, assays, and methods of the document can also be applied to other forms of pediatric cancer, including other brain tumors, e.g., anaplastic astrocytoma, atypical teratoid rhabdoid tumor (AT/RT), diffuse astrocytoma, ependymoma, glioblastoma multiforme (GBM), gliomas, myeloid leukemias, oligodendroglioma, pilocytic astrocytoma, and primitive neuroectodermal tumor (PNET).

In general, cancers suitable for treatment include those in which cancer cells express some level of functional p53, or in which functional p53 expression can be induced. For example, any cancer cell in which functional p53 is expressed but wherein the levels or activity of p53 are reduced in the cell by HDMX and/or HDMX can be beneficially treated using the compositions and methods disclosed herein. As disclosed herein, increases in p53 activity can lead to reduced viability or death of cancer cells in vitro and in vivo. Accordingly, compositions and methods disclosed herein can be used for the treatment of cancer. Agents suitable for use as HDMX and HDM2 modulating agents in the compositions and methods disclosed herein are disclosed herein.

In particular, Ewing sarcoma is a pediatric cancer of the bone and soft tissue that affects children and young adults. While a large number of patients shows a good initial response to multidisciplinary treatment, the subset of patients with metastatic and relapsed disease faces a poor prognosis, creating a need for new approaches to treatment.

Several recent sequencing efforts revealed remarkably quiet genomes in Ewing

sarcoma tumors [26]-[28]. Besides EWS/FLI, the oncogenic transcription factor that drives the disease, there are few recurrent mutations. Intriguingly, this is also true for TP53, one of the most commonly mutated genes in other cancers. The finding that the majority of Ewing sarcoma tumors present with functional p53 makes the negative regulator proteins HDM2 and HDMX (also expressed in Ewing sarcoma) feasible targets for therapy.

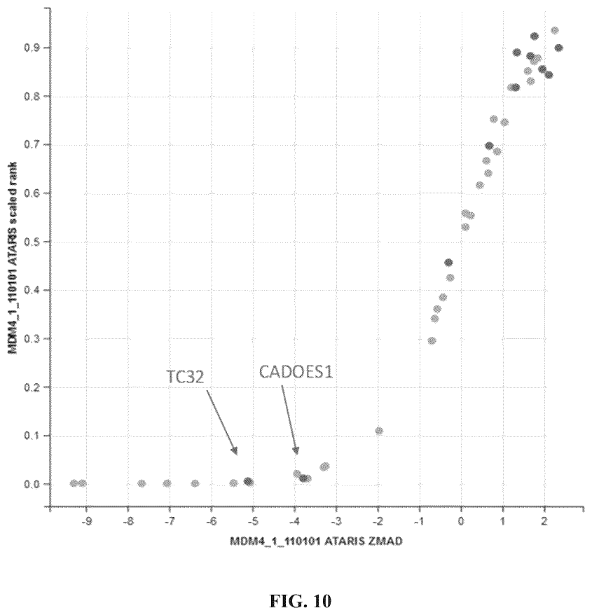

Given that the majority of Ewing sarcoma tumors are TP53 wild type, the dual-inhibition of HDM2 and HDMX is a promising treatment approach to Ewing sarcoma. This promise is strongly supported by screening data from a genome-wide CRISPR screen that included a number of Ewing sarcoma cell lines. Both HDM2 and HDMX were high-scoring dependencies exclusively in TP53 wild type cell lines (FIG. 9 and FIG. 10). Of note, the incidence of TP53 mutations is much higher in Ewing sarcoma cell lines than in primary Ewing sarcoma tumors.

The present disclosure includes treatment methods for pediatric cancer, e.g., methods for treating cancer in a pediatric subject (e.g., a human subject). As used herein, “treatment” means any manner in which one or more of the symptoms of a disease or disorder (e.g., pediatric cancer) are ameliorated or otherwise beneficially altered. As used herein, amelioration of the symptoms of a particular disorder (e.g., pediatric cancer) refers to any lessening, whether permanent or temporary, lasting or transient that can be attributed to or associated with treatment by the compositions and methods of the present invention. In some embodiments, treatment can promote or result in, for example, a decrease in the number of pediatric cancer cells (e.g., in a subject) relative to the number of the cancer cells (e.g., in the subject) prior to treatment; a decrease in the viability (e.g., the average/mean viability) of cancer cell(s) (e.g., in a subject) relative to the viability (e.g., the average/mean viability) of cancer cell(s) (e.g., in the subject) prior to treatment; a reduction in tumor size relative to tumor size prior to treatment; and/or reductions in one or more symptoms associated with one or more cancers in a subject relative to the subject's symptoms prior to treatment.

In some embodiments, the methods can include selecting a subject in need of treatment (e.g., a subject at risk for, that has, or that is suffering from, one or more pediatric cancers) and administering to the subject an effective dose of one or more of: (1) one or more PTAIBs; (2) one or more compositions and/or methods for inducing p53 expression and/or activity, including any combination of (1) with (2)) under conditions and for a period of time sufficient to treat the subject. Such methods can also include monitoring or evaluating the subject during and after administration of the composition to determine the efficacy of the treatment, and, if necessary, adjusting treatment (e.g., by altering the composition, by increasing the dose of a single administration of the composition, by increasing the number of doses of the composition administered per day, and/or by increasing the number of days the composition is administered) to improve efficacy.

In some embodiments, the methods can include developing a personalized treatment regimen for a pediatric subject with cancer. Such methods can include, e.g., identifying a pediatric subject with cancer cells that are sensitive to ICL PTAIBs and treating the subject with one or more ICL PTAIBs. In some embodiments, the methods can include determining the most appropriate treatment for a subject confirmed to have cancer (e.g., by determining the susceptibility of one or more of the subject's cancer cells to treatment using the compositions disclosed herein (e.g., in vitro)), developing a treatment regimen for the subject, and optionally administering to the subject a composition in accordance with the treatment regimen. These methods can include, for example:
(i) selecting a pediatric subject having a pediatric cancer; evaluating (e.g., detecting) the expression and/or activity of p53 in the subject's cancer (e.g., in a cancer cell obtained from the subject (e.g., obtained by biopsy); and, if p53 expression and/or activity is detected, providing the subject with a personalized treatment regimen that includes administering an effective amount of one or more ICL PTAIBs to the subject. In some embodiments, the method includes administering the one or more ICL PTAIBs to the pediatric subject under conditions and for a period of time sufficient to treat the subject;
(ii) selecting a pediatric subject having cancer; detecting the presence and/or level of a p53-HDMX complex in a sample (e.g., a cancer cell) obtained from the subject (e.g., a cancer cell obtained by biopsy); and, if the p53-HDMX complex is detected, providing the subject with a personalized treatment regimen that includes administering an effective amount of one or more ICL PTAIBs to the subject. In some embodiments, the method includes administering the one or more ICL PTAIBs to the pediatric subject under conditions and for a period of time sufficient to treat the subject;
(iii) selecting a pediatric subject having cancer; detecting the presence and/or level of a p53-HDMX complex in a sample (e.g., a cancer cell) obtained from the subject (e.g., a cancer cell obtained by biopsy) and assessing the level of p53 in the sample to determine if the level or activity of p53 is low (e.g., relative to the level or activity of p53 in a cancer cell that exhibits reduced viability when contacted with one or more ICL PTAIBs. In some embodiments the level of p53 is compared to the level of p53 in a JEG-3 and/or MCF-7 cell or cells. In some embodiments, activity can be assessed by titrating dissociation of HDMX-p53 complexes, as described herein); and, if the p53-HDMX complex is detected and the level of p53 is low, providing the subject with a personalized treatment regimen that includes administering an effective amount of one or more ICL PTAIBs to the subject. In some embodiments, the methods can also include providing the subject with a personalized treatment regimen that further includes administering an effective amount of a composition and/or method for inducing p53 expression and/or activity. In some embodiments, the method includes administering the one or more ICL PTAIBs and, optionally, the composition and/or method for inducing p53 expression and/or activity to the subject under conditions and for a period of time sufficient to treat the subject; and/or
(iv) selecting a pediatric subject with a pediatric cancer that has previously received one or more HDM2 modulating agents (e.g., Nutlin-3), but whose cancer cells were resistant (e.g., partially resistant) to the HDM2 modulating agents (e.g., Nutlin-3); and providing the subject with a personalized treatment regimen that includes administering an effective amount of one or more ICL PTAIBs and, optionally, a composition and/or method for inducing p53 expression and/or activity. In some embodiments, the method includes administering the one or more ICL PTAIBs and, optionally, the composition for inducing p53 expression and/or activity to the subject under conditions and for a period of time sufficient to treat the subject.

It should be noted that methods (i)-(iv) can be performed independently or together and in any order. Any of methods (i)-(iv) can also include monitoring or evaluating the subject during and after administration of the composition to determine the efficacy of the treatment, and, if necessary, adjusting treatment (e.g., by altering the composition, by increasing the dose of a single administration of the composition, by increasing the number of doses of the composition administered per day, and/or by increasing the number of days the composition is administered) to improve efficacy.

In some embodiments, ICL PTAIBs described herein can be used in the treatment of a subject in combination with other anti-cancer therapies or therapeutic methods. For example, ICL PTAIBs herein can be used in combination with chemotherapy, radiation therapy/radiotherapy, hormone, and immunotherapy such as antibody therapy.

The term “subject” is used throughout the specification to describe a pediatric animal, human or non-human, to whom treatment according to the methods of the present invention is provided. As used herein, the terms “cancer”, “hyperproliferative” and “neoplastic” refer to cells having the capacity for autonomous growth, i.e., an abnormal state or condition characterized by rapidly proliferating cell growth. As described herein, the present methods can be used to treat any pediatric cancer cell capable of expressing functional p53. For example, any pediatric cancer cell in which functional p53 is expressed but wherein the levels or activity of p53 are reduced in the cell by HDMX and/or HDMX can be beneficially treated using the compositions and methods disclosed herein. Wild-type and/or fully functional p53 activity is not required. For example, pediatric cancer cells which express mutant p53 that retains some function can be beneficially treated.

Accordingly, the present disclosure can include: (1) identifying a human pediatric subject with a pediatric cancer; and (2) determining if the subject's cancer cells encode or express functional p53; and (3) treating the subject or developing a treatment for the subject if the subject's cancer cells express functional p53 using the compositions and methods disclosed herein. For example, p53 function can be assessed in any of the cancers below.

In some instances, a subject or a cell from a subject should be capable of expressing functional p53. Such functional p53 should have some p53 function but does not have to have the same level of function as wild type p53. Accordingly, functional p53 can include mutated p53 that retains some level of function. In some instances, functional p53 can have 10%, 20%, 30%, 40%, 50%, 60%, 70%, 80%, 90%, 95%, or more of the functional activity of wild-type and/or fully functional p53 (e.g., wild-type and/or fully functional p53 in a non-cancer cell from the same subject). In some embodiments, a cell may be capable of expressing functional p53 but functional p53 is not detectable (for example, functional p53 is expressed but rapidly degraded in the cell). Such cells can be identified by detecting that the cell encodes functional p53. Such methods can be performed, e.g., using, e.g., DNA probes and/or by detecting p53 mRNA in the cell or a sample therefrom.

Methods for identifying a pediatric subject at risk for developing and/or with pediatric cancer are known in the art. For example, methods for identifying a pediatric subject at risk for developing pediatric cancer (e.g., a subject with an increased likelihood for developing cancer) are known in the art (see, e.g., U.S. Pat. No. 7611870 and Jie Li et al., Nature, Identification of high-quality cancer prognostic markers and metastasis network modules (2010)). Exemplary methods for identifying a subject with cancer are also known in the art and include self-evaluation, clinical evaluation (including physical examination and biopsy), laboratory analysis (e.g., biomarker analysis), the Papanicolaou test (Pap smear), and imaging methods (e.g., mammography, MRI, PET and/or CT scan and angiogram). In some embodiments, the p53-HDMX biomarker disclosed herein is used to identify a pediatric subject with a pediatric cancer (e.g., pediatric cancer that is susceptible to treatment with an ICL PTAIB).

As used herein, p53 activity can include, but is not limited, for example, p53 transcriptional activity (which can be assessed, e.g., by monitoring the transcription, mRNA levels, or protein levels of a target of p53, e.g., a p53 transcriptional target. Suitable p53 transcriptional targets are known in the art and include, but are not limited to, e.g., SFN, GADD45A, CRYZ, S100A2, BTG2, ODC1, TP53I3, TGFA, PCBP4, PLK2, CDC25C, CCNG1, IER3, TAP1, CDKN1A, EEF1A1, THBS2, ANLN, IGFBP3, EGFR, HGF, SERPINE1, MET, NOS3, TNFRSF10B, SCARA3, RRM2B, GML, DKK1, FAS, SCD, LRDD, CTSD, CD82, HSPA8, P53AIP1, SLC38A2, MDM2, HDM2, RB1, BDKRB2, MMP2, CX3CL1, SERPINB4, GDF15, BBC3, BAX, PCNA, TRPM2, and P2RXL1) and/or p53 functional activity (e.g., p53 protein-interaction based function, e.g., cell death (e.g., necrosis and apoptosis), and cell cycle arrest). p53 activity can also be assessed by determining p53 transcription, mRNA, or protein levels. Methods for carrying out each of these exemplary methods are generally known in the art.

### Kits

The compounds and pharmaceutical compositions described herein can be provided in a kit. For example, the kit can include compositions and methods for developing a personalized treatment method for a subject with cancer. In some embodiments, these kits can include compositions for detecting a biomarker of p53 in complex with HDMX (e.g., an antibody that binds specifically to the complex and/or components required to immunoprecipitate p53 or HDMX and to detect p53 or HDMX by immunoblotting (e.g., a kit can provide a first antibody (e.g., an anti-p53 antibody) to immunoprecipitate p53 and a second antibody (e.g., an anti-HDMX antibody) to detect HDMX by immunoblotting; or a kit can provide a first antibody (e.g., an anti-HDMX antibody) to immunoprecipitate HDMX and a second antibody (e.g., an anti-p53 antibody) to detect by immunoblotting). In some embodiments, the kit can further include compositions, including pharmaceutical compositions, that include: (1) one or more PTAIBs (e.g., SAH-p53-8); and/or (2) one or more compositions and/or methods for inducing p53 expression and/or activity, including any combination of (1)-(2) for administering to the subject. In such instances, the compositions for administering to the subject can be personalized to the subject. Alternatively, the compositions for administering to the subject are not personalized. In some embodiments, the compositions and methods for developing a personalized treatment method and the compositions for administering to the subject are provided in separate and independent kits.

The kits can also include informational material relevant to the compositions and methods of using the compositions. The informational material can be descriptive, instructional, marketing or other material that relates to the methods described herein and/or to the use of the agent for the methods described herein. For example, the informational material relates to the use of the compound to treat a subject who has, or who is at risk for developing cancer. The kits can also include paraphernalia for administering one or more compounds to a cell (in culture or in vivo) and/or for administering a cell to a patient, and any combination of the methods described herein.

In one embodiment, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In another embodiment, the informational material can include instructions to administer the pharmaceutical composition to a suitable subject, e.g., a pediatric human, e.g., a pediatric human having, or at risk for developing a pediatric cancer.

The informational material of the kits is not limited in its form. In many cases, the informational material (e.g., instructions) is provided in printed matter, such as in a printed text, drawing, and/or photograph, such as a label or printed sheet. However, the informational material can also be provided in other formats, such as Braille, computer readable material, video recording, or audio recording. Of course, the informational material can also be provided in any combination of formats.

In addition to the compound, the composition of the kit can include other ingredients, such as a solvent or buffer, a stabilizer, a preservative, and/or a second agent for treating a condition or disorder described herein. Alternatively, the other ingredients can be included in the kit, but in different compositions or containers than the compound. In such embodiments, the kit can include instructions for admixing the agent and the other ingredients, or for using one or more compounds together with the other ingredients.

The kit can include one or more containers for the pharmaceutical composition. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the pharmaceutical composition. For example, the kit can include a plurality of syringes, ampoules, foil packets, or blister packs, each containing a single unit dose of the pharmaceutical composition. The containers of the kits can be air tight and/or waterproof, and the containers can be labeled for a particular use. For example, a container can be labeled for use to treat a hearing disorder.

As noted above, the kits optionally include a device suitable for administration of the composition (e.g., a syringe, pipette, forceps, dropper, swab, or any such delivery device).

Thus, this disclosure provides insight into a precision medicine approach for reactivating cell death in a large group of pediatric cancers based on targeting HDM2/HDMX in the context of wild-type or functional p53 status.

### EXAMPLES

### Example 1: HDM2 and HDMX Expression in Cancer Cell Lines

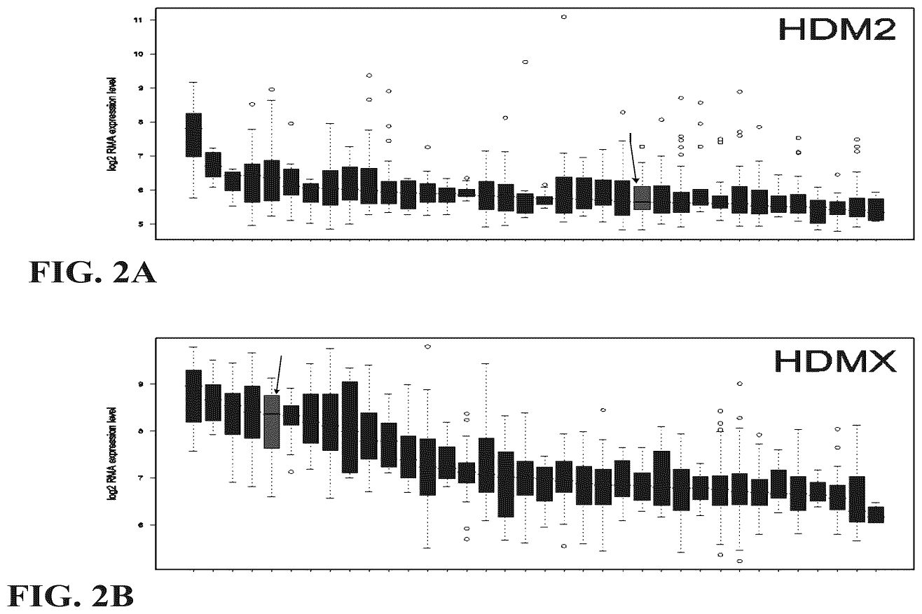

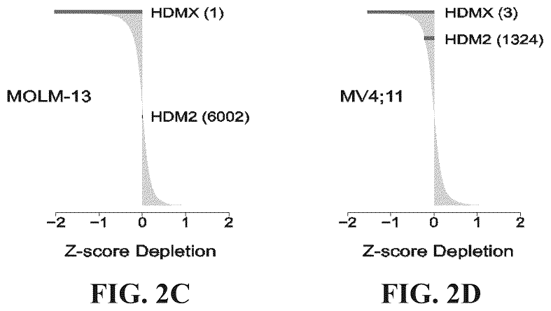

To better establish the relevance of HDM2 and HDMX expression across adult and pediatric cancers, the Cancer Cell Line Encyclopedia (CCLE) of the Broad Institute of Harvard and MIT, a resource offering genomic profiling of 1036 cancer cell lines of more than 20 different tissue types, including AML, was mined [13]. Whereas HDM2 has a relatively consistent level of expression across a diverse panel of cancer cell lines, with high levels observed in leukemias (FIG. 2A), HDMX exhibits more variable expression across cancer cell lines, with AML exhibiting among the highest expression of HDMX (FIG. 2B). In fact, of the leukemia cell lines interrogated, HDMX (also known as MDM4) ranked in the top 10 of all cancer genes in 32% of the lines. Thus, targeting both HDM2 and HDMX in pediatric myeloid leukemias, which are among the most challenging leukemias to treat in children, could reactivate p53 activities, including p53-mediated apoptosis.

Given the importance of a functional p53 signal transduction pathway to combat cancer, small molecule compounds (e.g., Nutlin-3) that block HDM2 have been developed [14]. However, such agents have generally been found to be ineffective in cancers that overexpress HDMX [15, 16, 18]. Thus, any therapeutic approach aimed at fully reactivating the p53 pathway in pediatric leukemias and other pediatric cancers that retain functional p53 coincident with HDM2 and HDMX expression must also simultaneously address HDMX [15, 17]. Indeed, in our functional genomic shRNA screen using 54,020 barcoded shRNAs targeting 11,194 genes, leukemia cell lines appeared to be more dependent on HDMX than HDM2. For example, HDMX scored in the top 3 gene dependencies for two AML cell lines with functional p53 (FIG. 2C, FIG. 2D).

We previously discovered that dual elevation of wild-type p53 and HDMX, in the form of a co-immunoprecipitated complex, was both a biomarker for resistance to selective HDM2 inhibition and the ideal biochemical setup for reactivating p53-mediated apoptosis

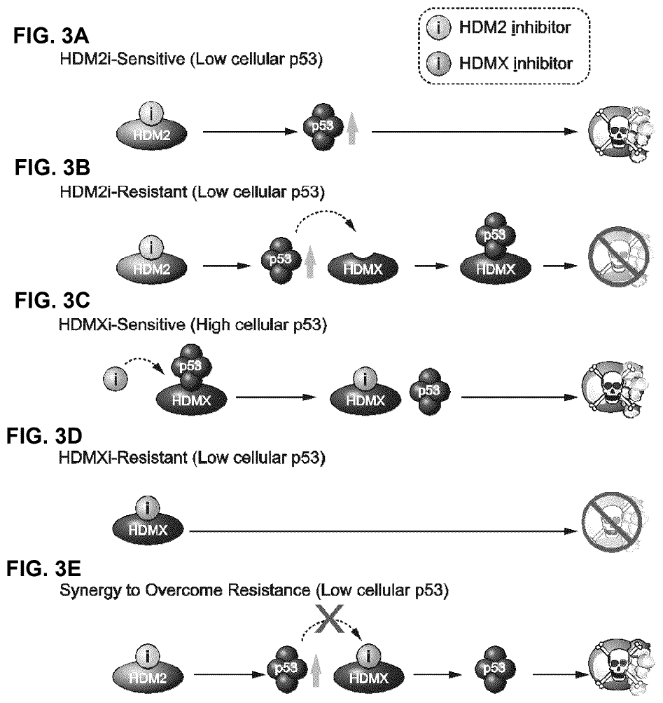

upon HDMX targeting (FIG. 3A-FIG. 3E). That is, the surge in p53 induced by HDM2 inhibition is neutralized by HDMX-mediated p53 sequestration (FIG. 3B), yet the capacity to target HDMX unleashes an arsenal of premade wild-type p53 to activate apoptosis (FIG. 3C).

We have now found (see, e.g., FIG. 2A and FIG. 2B) that AML cells express especially high levels of HDMX in the context of wild-type p53. Thus, a hypothetical dual HDM2/HDMX inhibitor could potentially be highly efficacious in reactivating p53-mediated apoptosis in pediatric AML and other pediatric cancers that retain functional p53 coincident with HDM2 and/or HDMX expression. Indeed, individualized patient selection for such a targeted therapy could be readily achieved by first determining wild-type versus mutant versus deletional status of the p53 gene, and for those patients with wild-type p53 status, an HDM2 and HDMX ELISA assay performed on cell lysates would identify an operational anti-apoptotic p53-HDM2-HDMX axis ideally suited for targeted treatment (FIG. 3E).

### Example 2: Structural Models of HDM2 and HDMX Complexed With Hydrocarbon-Stapled Peptides

Whereas small molecules are most effective at targeting small and deep hydrophobic clefts, such as in enzyme targets, the broad, shallow, and complex interfaces of protein interactions present a formidable challenge. We have harnessed the natural complexity and bioactive structure of alpha-helical protein interaction motifs to generate hydrocarbon-stapled peptides for therapeutic targeting. Drawing on structural data regarding the p53-HDM2 and p53-HDMX complexes [9], we have developed internally cross-linked (ICL) p53 transactivation domain-based inhibitor peptides (PTAIBs) targeting HDM2 and HDMX modeled after the alpha-helical transactivation domain of p53 and validated these

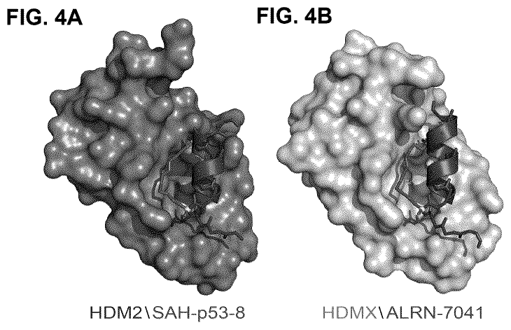

novel agents as inhibitors of both p53-HDM2 and p53-HDMX interactions (FIG. 4). For example, a particular ICL PTAIB, SAH-p53-8, can target HDM2 and HDMX in cells, block the formation of p53-HDM2 and p53-HDMX complexes, and thereby restore the p53

pathway [18]. Likewise, SAH-p53-8 suppresses tumor growth in a mouse model of HDM2/HDMX-overexpressing choriocarcinoma by triggering the upregulation of p53 transcriptional targets [18].

### Example 3: Potent and Sequence-Dependent Binding of SAH-p53-8 for HDMX

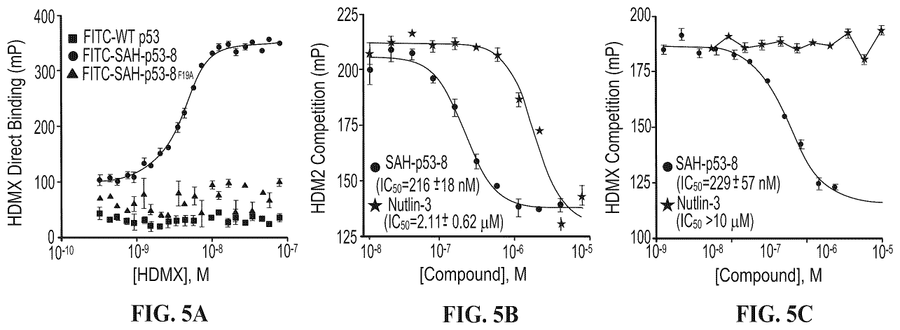

In contrast to the corresponding unmodified p53 peptide, our stapled analog, SAH-p53-8, demonstrated remarkably high (i.e., nanomolar) affinity for HDMX (K_{D}=2.3 nM); importantly, alanine mutagenesis of the critical F19 residue effectively completely abrogated binding activity, highlighting the specificity of the interaction (FIG. 5A).

We then performed competition binding assays to test the capacity of SAH-p53-8 to disrupt the high affinity complexes between FITC-SAH-p53-8 and HDM2 and HDMX. In contrast to the small molecule inhibitor, Nutlin-3, which only disrupted the HDM2 interaction, SAH-p53-8 potently dissociated both complexes (FIG. 5B, FIG. 5C).

### Example 4: Creation of ALRN-7041

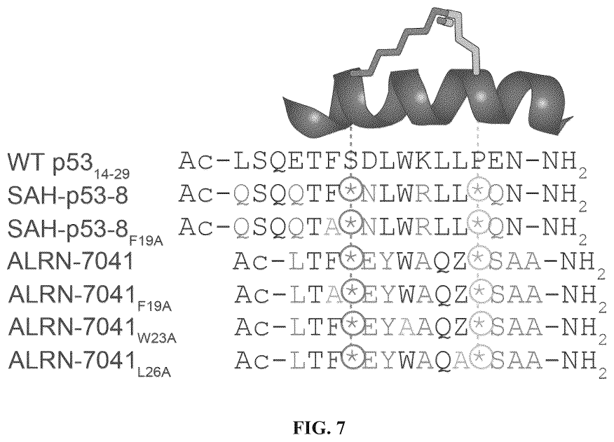

SAH-p53-8 was further modified to create ALRN-7041, which possessed improved drug-like properties (including improved stability, decreased serum binding, increased cellular uptake in the presence of serum, etc.) for targeting HDM2 and HDMX in cells and in vivo [20]. Although related to SAH-p53-8, ALRN-7041 contains significant modifications (FIG. 7). From the original 14 amino acids constituting SAH-p53-8, 12 amino acids were added, removed, or modified to generate ALRN-7041 (a peptide with a total of 12 amino acids: LTF*EYWAQZ*SAA, wherein *denotes the location of a hydrocarbon staple; and Z is Cba; SEQ ID NO:7), yet the critical HDM2 and HDMX interacting residues or non-natural analogs thereof were preserved (e.g. F19, W23, L26 mimetic). Many of these modifications were not obvious, yet confer significantly improved pharmaceutical properties (as described above) while maintaining the ability to disarming HDM2 and HDMX.

### Example 5: Dose-Responsive Activity of ALRN-7041 on Pediatric Cancer Cells

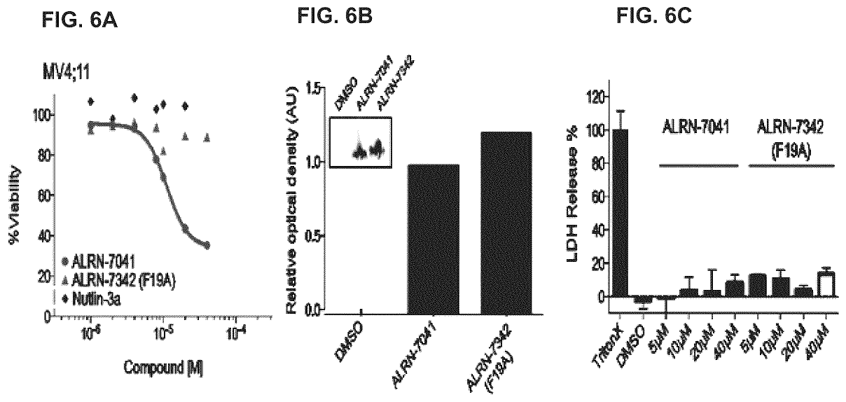

ALRN-7041 and its analogs are taken up by pediatric AML and other pediatric cancer cells in the presence of full serum and impairs cell viability in both a dose-responsive and peptide sequence-dependent fashion (FIG. 6A, FIG. 6B).

Importantly, in sharp contrast, Nutlin-3a, which only inhibits HDM2, has little to no effect on pediatric AML or other pediatric cancer cells that co-express HDMX. These data highlight the potential of our dual HDM2/HDMX targeting strategy to restore the p53-mediated cell death pathway in pediatric AML and other pediatric cancers that retain functional p53 coincident with HDM2 and/or HDMX expression.

### Example 6: SAH-p53-8 Dissociates the Inhibitory p53/HDMX Complex in Tumor Cells

Using immunoprecipitation and a Western blot assay it was demonstrated that SAH-p53-8, but not Nutlin-3, can dissociate the inhibitory p53/HDMX complex in solid tumor cells (FIG. 8).

### Example 7: Selective Susceptibility of Pediatric Leukemia Cell Lines to ALRN-7041 is Based on Wild-type p53 Expression

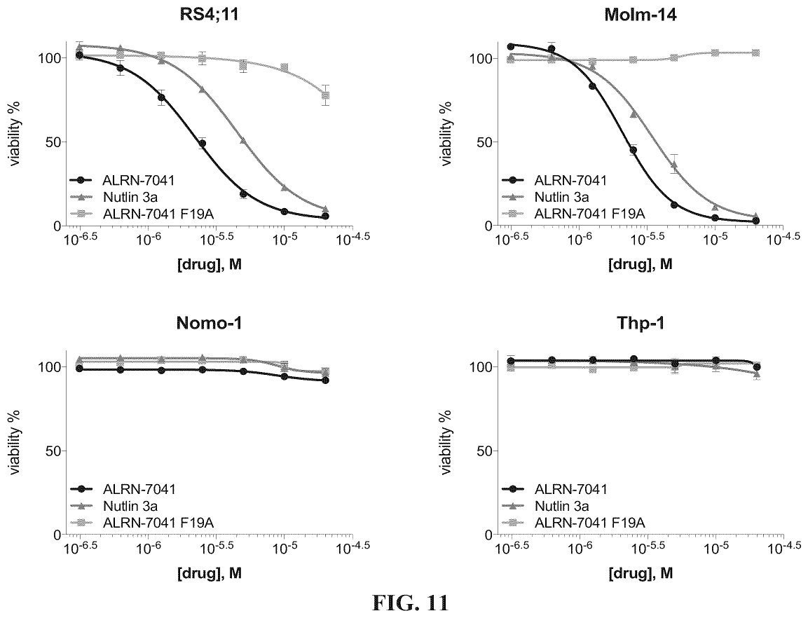

Cells were plated in 96-well opaque plates (2×10⁴/well) in RPMI containing 10% FBS and, the following day, the cells were treated with the indicated concentrations of drug or vehicle control (0.2% DMSO). Drug stocks (10, 5, 2.5, 1.25, 0.625, 0.313 mM in 100% DMSO) were diluted into ddH2O to achieve 10X working stocks of 200, 100, 50, 25, 12.5, 6.25, and 3.13 μM, which were then diluted 10-fold into the treatment wells. Cell viability was measured after 48 h by CellTiter-Glo assay (Promega, Madison, WI, USA), performed according to the manufacturer's instructions, and percent viability calculated based on the untreated controls. Error bars are mean±s.e.m for experiments performed in technical triplicate.

Whereas RS4; 11 and Molm-14 leukemia cells, which bear wild-type p53, succumb to ALRN-7041 in dose-responsive fashion, Nomo-1 and Thp-1, which express mutant forms of p53 (C242 and R174 frameshift mutant, respectively), are unaffected by treatment (see, FIG. 11). Importantly, the sensitivity of RS4; 11 and Molm-14 cells is peptide sequence dependent, as the F19A point mutant analog of ALRN-7041, ALRN-7041 F19A, has no effect (FIG. 11). Leukemia cells were treated in 10% serum with a serial dilution of stapled p53 peptides or Nutlin 3a, and then measured by CellTiter-Glo assay.

### Example 8: ALRN-7041 Dose-Responsively Upregulates p53 Protein Level in RS4; 11 Cells

RS4; 11 cells were plated in 6-well plates (10⁶/well) in RMPI containing 10% FBS and treated for 3 hours with ALRN-7041, ALRN-7041 F19A, or vehicle control (0.2% DMSO) in the presence of 10% serum. Cells were then harvested by centrifugation, washed in ice cold PBS, and resuspended in 100 viL PBS containing 1 μL Fixable Viability Stain 450 (BD Biosciences, Franklin Lakes. NJ, USA) and incubated in the dark at room temperature for 15 minutes. The cells were stained for p53 immunofluorescence with Cytofix/Cytoperm and the PE Mouse Anti-p53 Set (BD Biosciences) according to the manufacturer's instructions, using 10 viL PE-G59-12 anti-p53 mouse IgG1 or isotype control antibody per 10 6 cells. Stained and fixed cells were analyzed on an LSR II flow cytometer (BD Biosciences). Cell populations were gated on forward and side scatter to select for single cells and on fluorescence at 450 nm to select for live cells, and PE fluorescence in the gated population was measured at 578 nm.

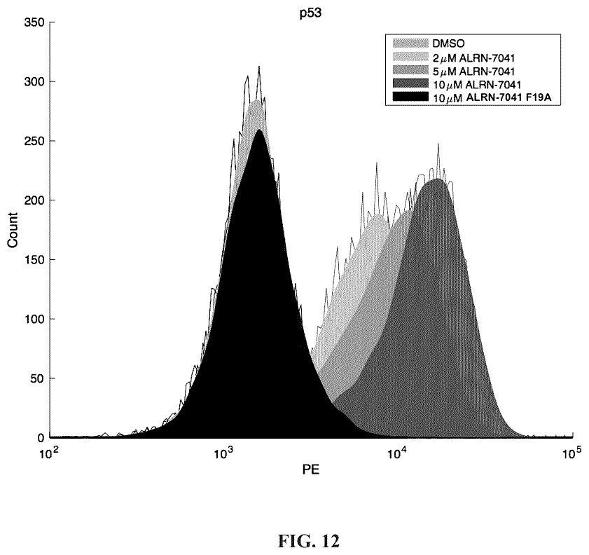

ALRN-7041 dose-responsively upregulates p53 protein level in RS4; 11 cells, as assessed by flow cytometry. The response is peptide sequence specific, as reflected by no effect of ALRN-7041 F19A on p53 protein level (FIG. 12).

### Example 9: Susceptibility of Pediatric Diffuse Interstitial Pontine Glioma (DIPG) Neurospheres to ALRN-7041

Cells were plated in 96-well opaque plates (5000 cells/well) in Neurobasal-A/DMEM/F-12 mixture media containing HEPES, MEM sodium pyruvate, MEM non-essential amino acids, GlutaMAX, B27 Supplement, H-EGF, H-FGF, H-PDGF-AA, H-PDGF-BB, and heparin. The following day, the cells were treated with the indicated concentrations of drug or vehicle control (0.2% DMSO). Drug stocks (10, 5, 2.5, 1.25, 0.625, 0.313 mM in 100% DMSO) were diluted into ddH2O to achieve 10× working stocks of 200, 100, 50, 25, 12.5, 6.25, and 3.13 μM, which were then diluted 10-fold into the treatment wells. Cell viability was measured after 72 h by CellTiter-Glo assay (Promega, Madison, WI, USA), performed according to the manufacturer's instructions, and percent viability calculated based on the untreated controls. Error bars are mean±s.e.m for experiments performed in technical triplicate.

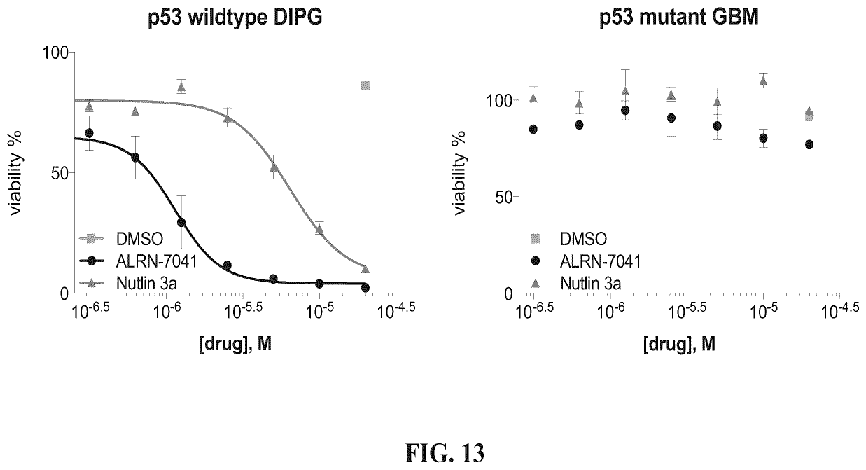

DIPG neurospheres bearing wild-type p53 succumb to treatment with ALRN-7041, with the stapled p53 peptide that targets both HDM and HDMX showing a markedly enhanced cytotoxic effect compared to a selective small molecule inhibitor of HDM2 (FIG. 13). As a measure of p53-dependence of the observed cytotoxic effect in DIPG, exemplary high grade glioma (e.g. GBM) neurospheres bearing mutant p53 show no susceptibility to HDM2 and HDMX targeting (FIG. 13). Neurospheres were treated with a serial dilution of stapled p53 peptide or Nutlin 3a, as measured by CellTiter-Glo assay.

### Example 10: Ewing Sarcoma Cell Lines Bearing Wild-type p53 are Selectively Susceptible to ALRN-7041 Treatment

Cells were plated in 96-well opaque plates and, the following day, the cells were treated with the indicated concentrations of drug or vehicle control (0.2% DMSO). Drug stocks (10, 5, 2.5, 1.25, 0.625, 0.313, 0.16, 0.078, 0.039 mM in 100% DMSO) were diluted into ddH2O to achieve 10X working stocks of 200, 100, 50, 25, 12.5, 6.25, 3.13, 1.56, 0.78, and 0.39 μM, which were then diluted 10-fold into the treatment wells. Cell viability was measured after 72 h by CellTiter-Glo assay (Promega, Madison, WI, USA), performed according to the manufacturer's instructions, and percent viability calculated based on the untreated controls. Error bars are mean±s.e.m. for eight technical replicates.

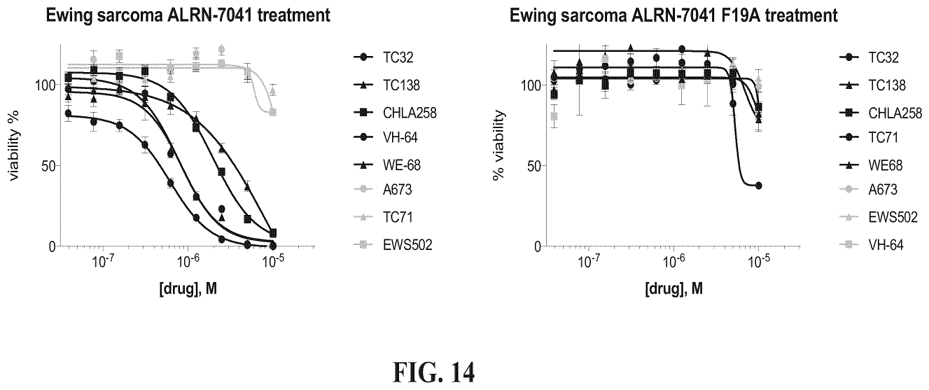

A panel of Ewing sarcoma cell lines that bear wild-type p53 (black) vs. mutant or deleted p53 (grey) were exposed to a serial dilution of ALRN-7041 (FIG. 14). ALRN-7041 dose-responsively impaired the viability of those Ewing sarcoma cells that maintained the expression of wild-type p53, but had little to no effect on Ewing sarcoma cells bearing mutant or deleted p53, as monitored by CellTiter-Glo assay (FIG. 14).

### Example 11: ALRN-7041 Reactivates the p53 Pathway in Ewing Sarcoma Lines Bearing Wild-Type p53

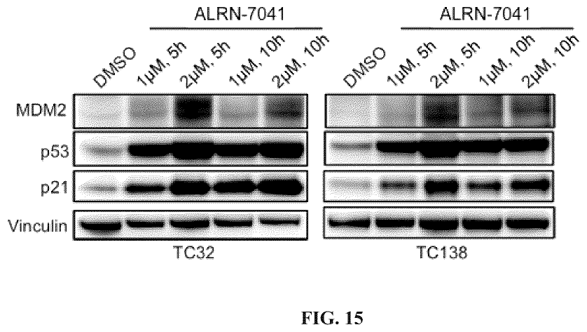

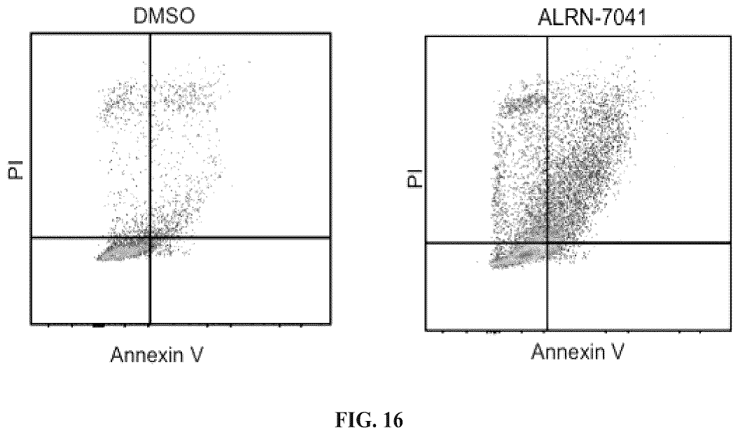

Western blot analysis of electrophoresed lysates from p53 wild-type TC32 and TC138 Ewing sarcoma cells treated with ALRN-7041 at the indicated doses and time points and probed with anti-MDM2, p53, and p21 antibodies. ALRN-7041 treatment causes robust induction of p53 and p21 and transient induction of MDM2, in the cell lines (FIG. 15).

### Example 12: ALRN-7041 Activates Apoptosis in an Ewing Sarcoma Cell Line Bearing Wild-type p53

TC32 Ewing sarcoma cells were treated with vehicle or 1 μM ALRN-7041 and then subjected to Annexin V/PI staining and FACS analysis at 48 hours post-treatment. ALRN-7041 induces robust Annexin V/PI double positivity in the treated TC32 Ewing sarcoma cells (FIG. 16).

### Example 13: Effect of ALRN-7041 treatment in Mice With a TC32 Ewing Sarcoma Xenograft

After TC32 Ewing sarcoma tumor engraftment, mice were treated with three intravenous doses of ALRN-7041 or vehicle and sacrificed 8 hours after the last dose. Each lane of the gel represents an individual mouse tumor, subjected to western analysis using anti-MDM2, p53, and p21 antibodies. Anti-tubulin was used as a loading control.

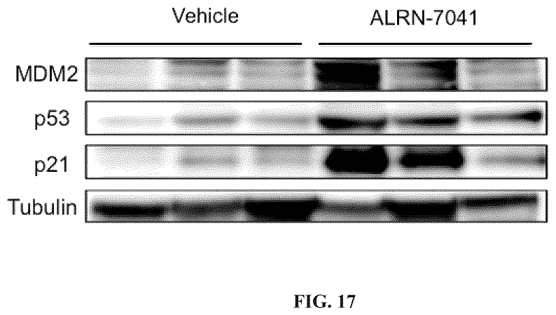

ALRN-7041 treatment of mice bearing a TC32 Ewing Sarcoma xenograft induces MDM2, p53, and p21 protein levels in tumor tissue (FIG. 17).

### Example 14: Effect of ALRN-7041 Treatment on MDM2 and p21 mRNA Levels in Tumor Tissue

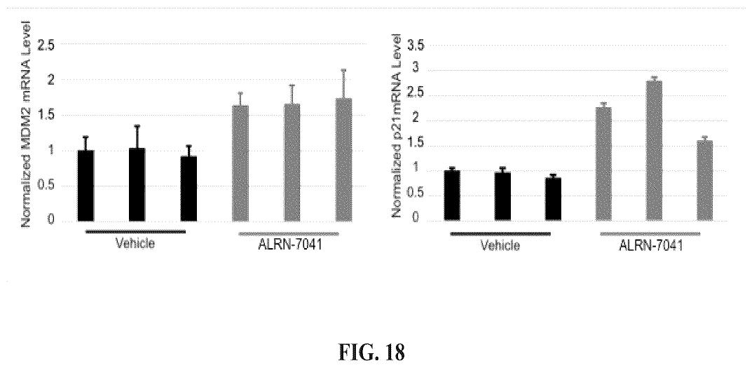

ALRN-7041 treatment of mice bearing TC32 Ewing Sarcoma xenografts increases MDM2 and p21 mRNA levels in tumor tissue (FIG. 18). Each bar represents the response of an individual mouse tumor, as assessed by replicates of mRNA quantitation. Values are normalized to vehicle-treated samples. Error bars are mean±SD.

### Example 15: ALRN-7041 Suppresses Tumor Growth

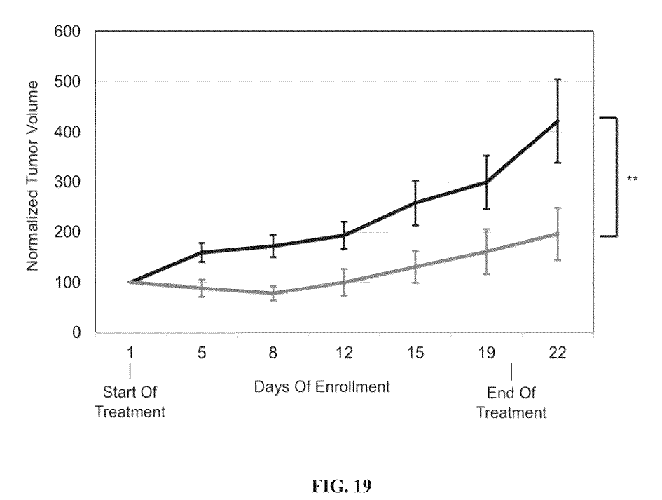

Treatment of mice bearing TC32 Ewing Sarcoma xenografts with 30 mg/kg ALRN-7041 IV q.o.d. (grey) or vehicle (black) demonstrates statistically significant suppression of tumor growth by the stapled p53 peptide (FIG. 19). Error bars are mean±SD. For each animal, treatment was instituted when tumors achieved a tumor volume of 100 mm³, as determined by caliper measurement. Statistical ignificance was calculated by two-way ANOVA analysis (p=0.0036).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Vousden, K. H. and Lane, D. P. p53 in health and disease. Nat Rev Mol Cell Biol 8, 275-283 (2007).
2. Kastan, M. B. et al. Participation of p53 protein in the cellular response to DNA damage. Cancer Res 51, 6304-6311 (1991).
3. Gasco, M. and Crook, T. p53 family members and chemoresistance in cancer: what we know and what we need to know. Drug Resist Updat 6, 323-328 (2003).
4. Nigro, J. M. et al. Mutations in the p53 gene occur in diverse human tumour types. Nature 342, 705-708 (1989).
5. Hollstein, M., Sidransky, D., Vogelstein, B. and Harris, C. C. p53 mutations in human cancers. Science 253, 49-53 (1991).
6. Honda, R., Tanaka, H. & Yasuda, H. Oncoprotein MDM2 is a ubiquitin ligase E3 for tumor suppressor p53. FEBS Lett 420, 25-27 (1997).
7. Moll, U. M. et al. Cytoplasmic sequestration of wild-type p53 protein impairs the G1 checkpoint after DNA damage. Mol Cell Biol 16, 1126-1137 (1996).
8. Wade, M., Wang, Y. V. and Wahl, G. M. The p53 orchestra: Mdm2 and Mdmx set the tone. Trends Cell Biol 20, 299-309 (2010).
9. Pazgier, M. et al. Structural basis for high-affinity peptide inhibition of p53 interactions with MDM2 and MDMX. Proc Natl Acad Sci USA 106, 4665-4670 (2009).
10. Tao, W. and Levine, A. J. Nucleocytoplasmic shuttling of oncoprotein Hdm2 is required for Hdm2-mediated degradation of p53. Proc Natl Acad Sci USA 96, 3077-3080 (1999).
11. Shvarts, A. et al. MDMX: a novel p53-binding protein with some functional properties of MDM2. EMBO J 15, 5349-5357 (1996).
12. Brown, C. J. et al. Reactivation of p53: from peptides to small molecules. Trends Pharm Sci 32, 53-62 (2011).
13. Barretina, J. et al. The cancer cell line encyclopedia enables predictive modelling of anticancer drug sensitivity. Nature 483, 603-607 (2012).
14. Vassilev, L. T. et al. In Vivo Activation of the p53 Pathway by Small-Molecule Antagonists of MDM2. Science 303, 844-848 (2004).
15. Laurie, N. A. et al. Inactivation of the p53 pathway in retinoblastoma. Nature 444, 61-66 (2006).
16. Xia, M. et al. Elevated MDM2 boosts the apoptotic activity of p53-MDM2 binding inhibitors by facilitating MDMX degradation. Cell Cycle 7, 1604-1612 (2008).
17. Gembarska, A. et al. MDM4 is a key therapeutic target in cutaneous melanoma. Nat Med 18, 1239-1247 (2012).
18. Bernal, F. et al. A stapled p53 helix overcomes HDMX-mediated suppression of p53. Cancer Cell 18, 411-422 (2010).
19. Bernal, F. et al. Reactivation of the p53 tumor suppressor pathway by a stapled p53 peptide. J Am Chem Soc 129, 2456-2457 (2007).
20. Chang, Y. S. et al. Stapled alpha—helical peptide drug development: A potent dual inhibitor of MDM2 and MDMX for p53-dependent cancer therapy Proc Natl Acad Sci USA (2013).
21. Bird, G. H., Bernal, F., Pitter, K. and Walensky, L. D. Synthesis and biophysical characterization of stabilized alpha-helices of BCL-2 domains. Methods Enzymol 446, 369-386 (2008).
22. Walensky, L. D. et al. Activation of apoptosis in vivo by a hydrocarbon-stapled BH3 helix. Science 305, 1466-1470 (2004).
23. Pitter, K., Bernal, F., Labelle, J. and Walensky, L. D. Dissection of the BCL-2 family signaling network with stabilized alpha-helices of BCL-2 domains. Methods Enzymol 446, 387-408 (2008).
24. van Delft, M. F. et al. The BH3 mimetic ABT-737 targets selective Bcl-2 proteins and efficiently induces apoptosis via Bak/Bax if Mcl-1 is neutralized. Cancer Cell 10, 389-399 (2006).
25. Bassan, R. and Hoelzer, D. Modern therapy of acute lymphoblastic leukemia. J Clin Oncol 29, 532-543 (2011).
26. Crompton, B. D. et al. The genomic landscape of pediatric Ewing sarcoma. Cancer Discov 4, 1326-1341 (2014).
27. Tirode, F. et al. Genomic landscape of Ewing sarcoma defines an aggressive subtype with co-association of STAG2 and TP53 mutations. Cancer Discov 4, 1342-1353 (2014).
28. Brohl et al. The genomic landscape of the Ewing sarcoma family of tumors reveals recurrent STAG2 mutation. PLoS Genet 10, e1004475 (2014).
29. Pfaff, E. et al. TP53 mutation is frequently associated with CTNNB1 mutation or MYCN
30. amplification and is compatible with long-term survival in medulloblastoma. J Clin Oncol 28, 5188-5196 (2010).
31. Parsons, D. W. et al. The genetic landscape of the childhood cancer medulloblastoma. Science 331, 435-439 (2011).
32. Saylors, R. L., 3rd et al. Infrequent p53 gene mutations in medulloblastomas. Cancer Res 51, 4721-4723 (1991).
33. Xu, J. et al. Pediatric brain tumor cell lines. J Cell Biochem 116, 218-224 (2015).
34. Ghassemifar, S. and S. M. Mendrysa. MDM2 antagonism by nutlin-3 induces death in human medulloblastoma cells. Neurosci Lett 513, 106-110 (2012).
35. Kunkele, A. et al. Pharmacological activation of the p53 pathway by nutlin-3 exerts antitumoral effects in medulloblastomas. Neuro Oncol 14, 859-869 (2012).
36. Carol, H. et al. Initial testing of the MDM2 inhibitor RG7112 by the Pediatric Preclinical Testing Program. Pediatr Blood Cancer 60, 633-641 (2013).
37. Pollack, I. F. et al. Age and TP53 mutation frequency in childhood malignant gliomas: results in a multi-institutional cohort. Cancer Res 61, 7404-7407 (2001).
38. Sung, T. et al. Preferential inactivation of the p53 tumor suppressor pathway and lack of EGFR amplification distinguish de novo high grade pediatric astrocytomas from de novo adult astrocytomas. Brain Pathol 10, 249-259 (2000).
39. Di Sapio, A. et al. Molecular genetic changes in a series of neuroepithelial tumors of childhood. J Neurooncol 59, 117-122 (2002).
40. Schiffer, D. et al. Mutations and immunohistochemistry of p53 and proliferation markers in astrocytic tumors of childhood. Childs Nery Syst 11, 517-522 (1995).
41. Schiffman, J. D. et al. Oncogenic BRAF mutation with CDKN2A inactivation is characteristic of a subset of pediatric malignant astrocytomas. Cancer Res 70, 512-519 (2010).
42. Pollack, I. F. et al. The relationship between TP53 mutations and overexpression of p53 and prognosis in malignant gliomas of childhood. Cancer Res 57, 304-309 (1997).
43. Rasheed, B. K. et al. Alterations of the TP53 gene in human gliomas. Cancer Res 54, 1324-1330 (1994).
44. Sengupta, S. et al. A study of histopathological spectrum and expression of Ki-67, TP53 in primary brain tumors of pediatric age group. Indian J Med Paediatr Oncol 33, 25-31 (2012).
45. Litofsky, N. S., D. Hinton, and C. Raffel. The lack of a role for p53 in astrocytomas in pediatric patients. Neurosurgery 34, 967-973 (1994).
46. Mendrysa, S. M., S. Ghassemifar, and R. Malek. p53 in the CNS: Perspectives on development, stem cells, and cancer. Genes Cancer 2, 431-442 (2011).
47. Grasso, C. S. et al. Functionally defined therapeutic targets in diffuse intrinsic pontine glioma. Nat Med 21, 555-559 (2015).
48. Ohtsubo C. et al. Cytoplasmic tethering is involved in synergistic inhibition of p53 by Mdmx and Mdm2. Cancer Sci 100, 1291-1299 (2009).
49. Richmond, J. et al. Effective targeting of the p53-MDM2 axis in preclinical models of infant MLL-rearranged acute lymphoblastic leukemia. Clin Cancer Res 21, 1395-1405 (2015).
50. Inomistova, M. V. et al. Prognostic significance of MDM2 gene expression in childhood neuroblastoma. Exp Oncol 37, 111-115 (2015).

1. A method of treating a pediatric cancer, the method comprising administering to a human subject with a pediatric cancer a therapeutically effective amount of one or more internally cross-linked (ICL) p53 transactivation domain-based inhibitor peptides (PTAIBs), the pediatric cancer comprising: (a) detectable wild-type p53; or (b) detectable functional p53.

2. (canceled)

3. The method of claim 1, wherein the pediatric cancer further comprises detectable HDM2 and/or HDMX.

4. The method of claim 3, wherein all or some of the detectable HDM2 and/or HDMX is complexed to wild-type or functional p53.

5. A method for predicting the efficacy of an internally cross-linked (ICL) p53 transactivation domain-based inhibitor peptide (PTAIB) in reversing the inhibition of p53 activity in a pediatric cancer, the method comprising:
a. testing a cell of a pediatric cancer for the presence of wild-type or functional p53, and
b. predicting that an ICL PTAIB that targets HDM2, HDMX, or HDM2 and HDMX would likely reverse inhibition of p53 activity in the cancer if the cell comprises wild-type or functional p53.

6. A method for predicting the efficacy of an internally cross-linked (ICL) p53 transactivation domain-based inhibitor peptide (PTAIB) in treating a pediatric cancer, the method comprising:
a. testing a cell of a pediatric cancer for the presence of wild-type or functional p53, and
b. predicting that an ICL PTAIB that targets HDM2, HDMX, or HDM2 and HDMX would likely reverse inhibition of p53 activity in the cancer and treat the cancer if the cell comprises wild-type or functional p53.

7. The method of claim 5, further comprising testing a cell of the pediatric cancer for the presence of HDM2 and/or HDMX, and predicting that an ICL PTAIB that targets HDM2, HDMX, or HDM2 and HDMX would likely reverse inhibition of p53 activity in the cancer if the cell comprises detectable wild-type or functional p53 and detectable HDM2 and/or HDMX.

8. The method of claim 7, wherein all or some of the detectable HDM2 and/or HDMX is complexed to wild-type or functional p53.

9. The method of claim 5, further comprising, if the cancer cell is found to express wild-type or functional p53, administering to the human subject with the pediatric cancer a therapeutically effective amount of one or more ICL PTAIBs that target HDM2 and/or HDMX.

10. The method of claim 5, further comprising, if the cancer cell is found to express wild-type or functional p53 and detectable HDM2 and/or HDMX, administering to the human subject with the pediatric cancer a therapeutically effective amount of one or more ICL PTAIBs that target HDM2 and/or HDMX.

11. The method of claim 10, wherein all or some of the detectable HDM2 and/or HDMX is complexed to wild-type or functional p53.

12. The method of claim 1, wherein the one or more administered ICL PTAIBs comprise one or more ICL PTAIBs that target HDM2 and/or HDMX.

13. The method of claim 1, wherein the administered ICL PTAIB is: (a) a stapled PTAIB; (b) SAH-p53-8; (c) ALRN-7041; (d) ALRN-6924; or (e) SP315.

14.-17. (canceled)

18. The method of claim 1, further comprising treating the human subject with one or more additional therapeutic regimens.

19. The method of claim 18, wherein the one or more additional therapeutic regimens are selected from the group consisting of surgery, chemotherapy, radiation therapy, hormone therapy, and immunotherapy.

20. The method of claim 1, wherein the pediatric cancer is: (a) a pediatric leukemia; (b) Ewing sarcoma; (c) selected from the group consisting of retinoblastoma, neuroblastoma, osteosarcoma, a glioma, medulloblastoma, rhabdomyosarcoma, Wilm's tumor, and a malignant rhabdoid tumor; (d) a relapsed cancer; or (e) refractory to one or more previous treatments.

21. The method of claim 20, wherein: (a) the pediatric leukemia is acute myeloid leukemia or acute lymphoblastic leukemia; (b) the acute lymphoblastic leukemia is a T cell lineage acute lymphoblastic leukemia or a B cell lineage acute lymphoblastic leukemia; (c) the rhabdomyosarcoma is alveolar or embryonal rhabdomyosarcoma; or (d) the glioma is a diffuse interstitial pontine glioma.

22.-28. (canceled)

29. The method of claim 1, wherein the pediatric cancer was refractory to one or more previous treatments.

30. The method of claim 6, further comprising testing a cell of the pediatric cancer for the presence of HDM2 and/or HDMX, and predicting that an ICL PTAIB that targets HDM2, HDMX, or HDM2 and HDMX would likely reverse inhibition of p53 activity in the cancer if the cell comprises detectable wild-type or functional p53 and detectable HDM2 and/or HDMX.

31. The method of claim 30, wherein all or some of the detectable HDM2 and/or HDMX is complexed to wild-type or functional p53.

32. The method of claim 6, further comprising, if the cancer cell is found to express wild-type or functional p53, administering to the human subject with the pediatric cancer a therapeutically effective amount of one or more ICL PTAIBs that target HDM2 and/or HDMX.

33. The method of claim 6, further comprising, if the cancer cell is found to express wild-type or functional p53 and detectable HDM2 and/or HDMX, administering to the human subject with the pediatric cancer a therapeutically effective amount of one or more ICL PTAIBs that target HDM2 and/or HDMX.

34. The method of claim 33, wherein all or some of the detectable HDM2 and/or HDMX is complexed to wild-type or functional p53.


SEQUENCE LISTING (13 sequences)

SEQ ID NO: 1 - protein, 393 aa, Homo sapiens [sequence omitted]

SEQ ID NO: 2 - protein, 16 aa, Homo sapiens
LSQETFSDLWKLLPEN

SEQ ID NO: 3 - protein, 16 aa, synthetic construct
  REGION 1..16: note = Description of Artificial Sequence: Syntheticpeptide
  REGION 1..16: note = N-term Ac
  MOD_RES 7: note = Any amino acid with all-hydrocarbon staple to position 14
  MOD_RES 14: note = Any amino acid with all-hydrocarbon staple to position 7
  REGION 1..16: note = C-term NH2
QSQQTFXNLWRLLXQN

SEQ ID NO: 4 - protein, 17 aa, synthetic construct
  REGION 1..17: note = Description of Artificial Sequence: Syntheticpeptide
  REGION 1..17: note = N-term Ac
  MOD_RES 4: note = Any amino acid available for crosslinking with position 11
  MOD_RES 11: note = Any amino acid available for crosslinking with position 4
  MOD_RES 17: note = D-Ala
  REGION 1..17: note = C-term NH2
LTFXAYWAQLXAAAAAA

SEQ ID NO: 5 - protein, 14 aa, synthetic construct
  REGION 1..14: note = Description of Artificial Sequence: Syntheticpeptide
  REGION 1..14: note = N-term Ac
  MOD_RES 4: note = Any amino acid with all-hydrocarbon staple toposition 11
  MOD_RES 10: note = Any leucine mimetic derived amino acid
  MOD_RES 11: note = Any amino acid with all-hydrocarbon staple toposition 4
  REGION 1..14: note = C-term NH2
LTFXEYWAQXXSAA

SEQ ID NO: 6 - protein, 14 aa, synthetic construct
  REGION 1..14: note = Description of Artificial Sequence: Syntheticpeptide
  MOD_RES 4: note = Any non-natural amino acid available for crosslinkingwith position 11
  MOD_RES 10: note = Any leucine mimetic derived amino acid
  MOD_RES 11: note = Any non-natural amino acid available for crosslinkingwith position 4
LTFXEYWAQXXSAA

SEQ ID NO: 7 - protein, 14 aa, synthetic construct
  REGION 1..14: note = Description of Artificial Sequence: Syntheticpeptide
  MOD_RES 4: note = Any amino acid with all-hydrocarbon staple toposition 11
  MOD_RES 10: note = Cyclobutylalanine
  MOD_RES 11: note = Any amino acid with all-hydrocarbon staple toposition 4
LTFXEYWAQXXSAA

SEQ ID NO: 8 - protein, 16 aa, Homo sapiens
  REGION 1..16: note = N-term Ac
  REGION 1..16: note = C-term NH2
LSQETFSDLWKLLPEN

SEQ ID NO: 9 - protein, 16 aa, synthetic construct
  REGION 1..16: note = Description of Artificial Sequence: Syntheticpeptide
  REGION 1..16: note = N-term Ac
  MOD_RES 7: note = Any amino acid with all-hydrocarbon staple to position 14
  MOD_RES 14: note = Any amino acid with all-hydrocarbon staple to position 7
  REGION 1..16: note = C-term NH2
QSQQTAXNLWRLLXQN

SEQ ID NO: 10 - protein, 14 aa, synthetic construct
  REGION 1..14: note = Description of Artificial Sequence: Syntheticpeptide
  REGION 1..14: note = N-term Ac
  MOD_RES 4: note = Any amino acid with all-hydrocarbon staple toposition 11
  MOD_RES 10: note = Cyclobutylalanine
  MOD_RES 11: note = Any amino acid with all-hydrocarbon staple toposition 4
  REGION 1..14: note = C-term NH2
LTFXEYWAQXXSAA

SEQ ID NO: 11 - protein, 14 aa, synthetic construct
  REGION 1..14: note = Description of Artificial Sequence: Syntheticpeptide
  REGION 1..14: note = N-term Ac
  MOD_RES 4: note = Any amino acid with all-hydrocarbon staple toposition 11
  MOD_RES 10: note = Cyclobutylalanine
  MOD_RES 11: note = Any amino acid with all-hydrocarbon staple toposition 4
  REGION 1..14: note = C-term NH2
LTAXEYWAQXXSAA

SEQ ID NO: 12 - protein, 14 aa, synthetic construct
  REGION 1..14: note = Description of Artificial Sequence: Syntheticpeptide
  REGION 1..14: note = N-term Ac
  MOD_RES 4: note = Any amino acid with all-hydrocarbon staple toposition 11
  MOD_RES 10: note = Cyclobutylalanine
  MOD_RES 11: note = Any amino acid with all-hydrocarbon staple toposition 4
  REGION 1..14: note = C-term NH2
LTFXEYAAQXXSAA

SEQ ID NO: 13 - protein, 14 aa, synthetic construct
  REGION 1..14: note = Description of Artificial Sequence: Syntheticpeptide
  REGION 1..14: note = N-term Ac
  MOD_RES 4: note = Any amino acid with all-hydrocarbon staple toposition 11
  MOD_RES 11: note = Any amino acid with all-hydrocarbon staple toposition 4
  REGION 1..14: note = C-term NH2
LTFXEYWAQAXSAA